(19) United States
(12) Patent Application Publication
(10) Pub. No.: US 2009/0257960 A1
(43) Pub. Date: Oct. 15, 2009

(54) NEUTRALIZED ACID GROUP-CONTAINING POLYMERS AND THE USE THEREOF

(75) Inventors: Son Nguyen Kim, Hemsbach (DE); Thomas Kaiser, Hassloch (DE)

Correspondence Address: CONNOLLY BOVE LODGE & HUTZ LLP, 1875 EYE STREET, N.W., SUITE 1100, WASHINGTON, DC, 20006, US

(73) Assignee: BASF Aktiengesellschaft, Ludwigshafen (DE)

(21) Appl. No.: 12/299,067

(22) PCT Filed: May 3, 2007
(86) PCT No.: PCT/EP07/54309

§ 371 Date: Oct. 30, 2008

(30) Foreign Application Priority Data
May 4, 2006 (EP) 06113520.8

(51) Int. Cl. A61K 8/02; A61K 47/30; C08G 63/91; A61Q 5/00

(52) U.S. Cl. 424/47; 514/772.3; 525/418

(57) ABSTRACT

The present invention relates to a polymer component which consists of one polymer or of a plurality of different polymers, comprises at least one polyurethane, where the polymer or at least one of the polymers has acid groups which are neutralized partially with at least one inorganic base and partially with at least one organic base, to cosmetic or pharmaceutical compositions which comprise such a polymer component, and to a method of modifying the mechanical properties of such a polymer component, in which, for the neutralization, at least one inorganic base and at least one organic base are used.

The present invention relates to a polymer component which consists of one polymer or of a plurality of different polymers, comprises at least one polyurethane, where the polymer or at least one of the polymers has acid groups which are neutralized partially with at least one inorganic base and partially with at least one organic base, to cosmetic or pharmaceutical compositions which comprise such a polymer component, and to a method of modifying the mechanical properties of such a polymer component in which, for the neutralization, at least one inorganic base and at least one organic base is used.

Cosmetically and pharmaceutically acceptable water-soluble or water-dispersible polymers are used widely in cosmetics and medicine. They are used, for example, quite generally as thickeners for diverse types of formulations, such as, for example, gels, creams or emulsions. For these applications, use is often made of water-soluble polymers with anionic functionalities, such as, for example, crosslinked polyacrylic acid. For hair cosmetics in particular, crosslinked polymers with film-forming properties are used as conditioners in order to improve the dry and wet combability, the feel to the touch, the shine and/or the appearance of the hair, and to impart antistatic properties to the hair.

Difficulties often arise with the provision of products with a complex profile of properties. Thus, there is a need for polymers for cosmetic and pharmaceutical compositions which have good film-forming properties, where the flexibility of the resulting films can be adjusted as desired. In addition, the polymers should combine as many of the following properties as possible:
besides the flexible properties of the films obtained with them, they should also contribute to setting of the hair,
they should have good adhesion to keratin surfaces, in particular hair,
they should have a good ability to be washed out,
they should have good compatibility with solvents and, in particular, good propellant compatibility for producing spray formulations,
they should have good compatibility with as many different cosmetic and pharmaceutical active ingredients and auxiliaries as possible (the products obtained with them should have stability, clarity and/or good rheological properties),
hair-treatment compositions based on these polymers should exhibit no “flaking” effect,
they should have conditioning properties and improve the sensory properties of the hair, e.g. impart suppleness and shine to it and, after drying, not be sticky, or only be slightly sticky.

WO 94/03510 describes the use of polyurethanes of
a) at least one compound which comprises two or more active hydrogen atoms per molecule,
b) at least one diol comprising acid or salt groups and
c) at least one diisocyanate
   with a glass transition temperature of at least 15° C. and acid numbers of from 12 to 150 or the salts of these polyurethanes in cosmetic preparations and as binders or coatings in pharmaceutical preparations.

EP-A-619 111 describes the use of polyurethanes based on organic diisocyanates, diols and 2,2-hydroxymethyl-substituted carboxylates of the formula

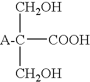

in which A is a hydrogen atom or a C₁-C₂₀-alkyl group, in hair-fixing compositions. At least some of the carboxylic acid groups here are neutralized with an organic or inorganic base. The diols here have a molecular weight in the range from 300 to 20 000; where, inter alia, polytetrahydrofurans are also specified as suitable diol component.

WO 94/13724 describes the use of cationic polyurethanes and polyureas of
(a) at least one diisocyanate which may have already been reacted beforehand with one or more compounds which comprise two or more active hydrogen atoms per molecule, and
(b) at least one diol comprising one or more tertiary, quaternary or protonated tertiary amine nitrogen atoms, primary or secondary amino alcohol, primary or secondary diamine or primary or secondary triamine
   with a glass transition temperature of at least 25° C. and an amine number from 50 to 200, based on the nonquaternized or protonated compounds, or those salts of these polyurethanes and polyureas as auxiliaries in cosmetic and pharmaceutical preparations.

WO 01/16200 describes a cosmetic composition comprising water-soluble or water-dispersible polyurethanes of an oligomer or polymer of
A) at least one diisocyanate,
B) at least one compound with at least two groups reactive toward isocyanate groups which are chosen from
   B1) aliphatic and cycloaliphatic polyols, polyamines and/or amino alcohols,
   B2) polyetherols and/or diaminopolyethers,
   B3) polysiloxanes with at least two active hydrogen atoms per molecule,
   B4) polyester polyols,
   and mixtures thereof, and
C) if appropriate at least one dicarboxylic acid and/or hydroxycarboxylic acid,
   where the oligomer comprises, per molecule, at least two urethane and/or urea groups and additionally at least two further functional groups which are chosen from hydroxyl groups, or primary and/or secondary amino groups.

EP-A-1 543 819 describes hair-treatment compositions based on elastic cationic polyurethanes.

EP-A-938 889 describes a cosmetic composition comprising at least one water-soluble or water-dispersible polyurethane of
a) at least one polymer with two active hydrogen atoms per molecule which is chosen from polytetrahydrofurans, polysiloxanes and mixtures thereof,
b) at least one polyesterdiol,
c) at least one compound with a molecular weight in the range from 56 to 300 which has two active hydrogen atoms per molecule,
d) at least one compound which has two active hydrogen atoms and at least one anionogenic or anionic group per molecule,
e) at least one diisocyanate,
   or the salts thereof, where the polyurethane comprises no unit originating from a primary or secondary amine which has an ionogenic or ionic group.

WO 99/58100 describes a cosmetic composition comprising at least one crosslinked, water-soluble or water-dispersible polyurethane of at least one polyurethane prepolymer with terminal isocyanate groups and at least one polymer with groups reactive toward isocyanate groups, where at least one of the components comprises a siloxane group.

DE-A-102 59 036 describes polyether urethanes containing allyl groups, polymers which comprise these in copolymerized form, and cosmetic or pharmaceutical compositions based on these polymers.

PCT/EP2005/013927 describes amine-containing polysiloxane urethane oligomers.

PCT/EP2005/008040 describes crosslinked polytetrahydrofuran-containing polyurethanes.

EP-A-957 119 describes crosslinked, water-soluble or water-dispersible polyurethanes of
A) at least one water-soluble or water-dispersible polyurethane prepolymer with terminal isocyanate groups of
   at least one compound with a molecular weight in the range from 56 to 300 which comprises two active hydrogen atoms per molecule,
   at least one polymer with two active hydrogen atoms per molecule,
   at least one compound which has two active hydrogen atoms and at least one ionogenic or ionic group per molecule,
   at least one diisocyanate,
B) at least one polymer with groups reactive toward isocyanate groups which are chosen from hydroxyl groups, primary and secondary amino groups and/or carboxyl groups,
   or the salts thereof, and the use of these polyurethanes as auxiliaries in cosmetics.

WO 03/085019 describes crosslinked polyurethanes based on polytetrahydrofuran and their use in cosmetic and pharmaceutical compositions.

EP-A-0 937 451 describes a hair-setting composition based on a water-soluble or water-dispersible polyurethane which comprises a 2,2-hydroxymethyl-substituted carboxylic acid diol in copolymerized form and is neutralized with a cosmetically acceptable organic or inorganic base.

WO 01/85821 describes polyurethanes based on at least one polyether with two active hydrogen atoms per molecule and their use for modifying rheological properties.

WO 01/10393, WO 01/10394 and WO 01/10397 describe cosmetics based on amphoteric urethane resins.

The object of the present invention is to provide novel film-forming polymers which are suitable for producing cosmetic and/or pharmaceutical compositions. Besides an overall good application profile for this field of use, as defined above, these polymers should have very good film-forming properties in particular. They should preferably enable their mechanical properties, in particular the elasticity and specifically the ratio of hardness to elasticity, to be adapted to the particular intended use.

Surprisingly, it has now been found that this object is achieved by a polymer component which consists of one polymer or of a plurality of different polymers, comprises at least one polyurethane and where the polymer or at least one of the polymers has acid groups which are neutralized partially with at least one inorganic base and partially with at least one organic base.

The invention therefore provides a cosmetic composition comprising
A) a polymer component which consists of one polymer or of a plurality of different polymers, comprises at least one polyurethane and where the polymer or at least one of the polymers has acid groups which are partially neutralized with at least one inorganic base and partially neutralized with at least one organic base, and
B) at least one cosmetically or pharmaceutically acceptable active ingredient or auxiliary.

The invention also provides a polymer component which consists of one polymer or of a plurality of different polymers, comprises at least one polyurethane, where the polymer or at least one of the polymers has acid groups which are neutralized partially with at least one inorganic base and partially with at least one organic base. This polymer component preferably comprises:
at least one polyurethane which has acid groups which are neutralized partially with at least one inorganic base and partially with at least one organic base, or
a mixture of at least one polyurethane which has acid groups which are neutralized at least partially with at least one inorganic base, and at least one polyurethane which has acid groups which are neutralized at least partially with at least one organic base.

The invention further provides a method of modifying the mechanical properties of a polymer component which consists of one polymer or of a plurality of different polymers, comprises at least one polyurethane and where the polymer or at least one of the polymers has acid groups, in which, for the neutralization, at least one inorganic base and at least one organic base are used.

For the purposes of the present invention, the expression alkyl comprises straight-chain and branched alkyl groups. Suitable short-chain alkyl groups are, for example, straight-chain or branched C₁-C₈-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc. Suitable longer-chain C₈-C₃₀-alkyl and C₈-C₃₀-alkenyl groups are straight-chain and branched alkyl and alkenyl groups. These are preferably predominantly linear alkyl radicals, as also arise in natural or synthetic fatty acids and fatty alcohols, and also oxo alcohols, which may, if appropriate, additionally be mono-, di- or polyunsaturated. These include, for example, n-hex(en)yl, n-hept(en)yl, n-oct(en)yl, n-non(en)yl, n-dec(en)yl, n-undec(en)yl, n-dodec(en)yl, n-tridec(en)yl, n-tetradec(en)yl, n-pentadec(en)yl, n-hexadec(en)yl, n-heptadec(en)yl, n-octadec(en)yl, n-nonadec(en)yl etc.

The expression “alkyl” also comprises substituted alkyl groups which can generally carry 1, 2, 3, 4, 5 or more than 5 substituents, preferably chosen from the groups cycloalkyl, aryl, hetaryl, acyl, NE¹E², NE¹E²E³⁺, COOH, carboxylate, —SO₃H, sulfonate and alkylaminocarbonyl.

For the purposes of the present invention, the expression “cycloalkyl” comprises unsubstituted and substituted cycloalkyl groups, preferably C₃-C₈-cycloalkyl groups, such as cyclopentyl, cyclohexyl or cycloheptyl, which in the case of a substitution, can generally carry 1, 2, 3, 4 or 5, preferably 1, 2 or 3 and particularly preferably 1, substituent(s), preferably chosen from the substituents specified for alkyl.

The expression “bicycloalkyl” is preferably a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like.

For the purposes of the present invention, the expression “heterocycloalkyl” comprises saturated, cycloaliphatic groups having generally 4 to 7, preferably 5 or 6, ring atoms, in which 1 or 2 of the ring carbon atoms are replaced by heteroatoms, preferably chosen from the elements oxygen, nitrogen and sulfur and which may be optionally substituted, where in the case of a substitution, these heterocycloaliphatic groups may carry 1, 2 or 3, preferably 1 or 2, particularly preferably 1, substituent(s) chosen from alkyl, aryl, COOR, COO⁻M⁺ and NE¹E², preferably alkyl. Examples of such hetero cycloaliphatic groups which may be mentioned are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

For the purposes of the present invention, the expression “aryl” comprises unsubstituted and substituted aryl groups and is preferably phenyl, tolyl, xylyl, mesityl, naphthyl, fluorenyl, anthracenyl, phenanthrenyl or naphthacenyl, particularly preferably phenyl or naphthyl, where these aryl groups, in the case of a substitution, can generally carry 1, 2, 3, 4 or 5, preferably 1, 2 or 3 and particularly preferably 1, substituent(s) chosen from the groups alkyl, alkoxy, carboxyl, carboxylate, —SO₃H, sulfonate, NE¹E², alkylene-NE¹E², nitro, cyano or halogen.

For the purposes of the present invention, the expression “hetaryl” comprises unsubstituted or substituted, heterocycloaromatic groups, preferably the groups pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl, where these heterocycloaromatic groups, in the case of a substitution, can generally carry 1, 2 or 3 substituents chosen from the groups alkyl, alkoxy, acyl, carboxyl, carboxylate, —SO₃H, sulfonate, NE¹E², alkylene-NE¹E² or halogen.

The above explanations of the expressions “alkyl”, “cycloalkyl”, “aryl”, “heterocycloalkyl” and “hetaryl” apply correspondingly to the expressions “alkoxycarbonyl”, “alkylaminocarbonyl”, “hydroxyalkyl”, “alkoxy”, etc.

For the purposes of the present invention, the expression “acyl” is alkanoyl or aroyl groups having generally 2 to 11, preferably 2 to 8, carbon atoms, for example the acetyl, propanoyl, butanoyl, pentanoyl, hexanoyl, heptanoyl, 2-ethylhexanoyl, 2-propylheptanoyl, benzoyl or naphthoyl group.

The groups NE¹E² are preferably N,N-dimethylamino, N-ethyl-N-methylamino, N,N-diethylamino, N,N-dipropylamino, N,N-diisopropylamino, N,N-di-n-butylamino, N,N-di-t-butylamino, N,N-dicyclohexylamino or N,N-diphenylamino.

The polymer component according to the invention and used according to the invention can consist of one or more polymers. If the polymer component consists only of one polymer, then it is a polyurethane which has acid groups which are neutralized partially with at least one inorganic base and partially with at least one organic base. If the polymer component consists of a plurality of polymers, then at least one of these polymers is a polyurethane. If the polymer component consists of a plurality of polymers, then at least one of these polymers has acid groups. The polymer having acid groups may be a polyurethane or a polymer different from polyurethanes. Preference is given to polymer components of a plurality of polymers which comprise at least one polyurethane having acid groups.

The polymer component according to the invention and used according to the invention can consist of one or more polymers which have acid groups. If the polymer component consists only of one polymer containing acid groups, then the acid groups of this one polymer are neutralized partially with at least one inorganic base and partially with at least one organic base. If the polymer component consists of a plurality of polymers containing acid groups, then (at least) one of these polymers can be neutralized partially with at least one inorganic base and partially with at least one organic base. If the polymer component consists of a plurality of polymers containing acid groups, then in one alternative embodiment, (at least) one of these polymers can be neutralized partially or completely with at least one inorganic base, and (at least) one other polymer can be neutralized partially or completely with at least one organic base.

The polymer component has preferably at least one polymer containing acid groups with an acid number in the range from 12 to 150, preferably 30 to 90.

The polymer component according to the invention and used according to the invention can comprise any further polymers provided the abovementioned conditions are met.

The acid groups may preferably be carboxylic acid groups, sulfonic acid groups, phosphonic acid groups and combinations thereof. Particular preference is given to carboxylic acid groups.

The acid groups of the polymer component may be partially or completely neutralized. Preferably, the acid groups of the polymer component are neutralized to at least 50%, particularly preferably to at least 75%, in particular to at least 95%. In one specific embodiment, the acid groups of the polymer component are completely neutralized.

Preferably, the organic base is chosen from organic amines.

Suitable amines are, for example, chosen from
primary amines of the formula 1.a

R¹—NH₂ (1.a)
in which R¹ is
a) alkyl which may be substituted by 1, 2, 3, 4 or more than 4 groups which are preferably chosen independently of one another from hydroxy, acyl, alkoxycarbonyl, alkylaminocarbonyl, cycloalkyl, heterocycloalkyl, aryl or hetaryl and/or the alkyl group may be interrupted by 1, 2, 3, 4 or more than 4 nonadjacent heteroatoms or groups containing heteroatoms which are preferably chosen from O and S,
b) cycloalkyl where these radicals may be substituted by 1, 2, 3, 4 or more than 4 of the following groups, which are preferably chosen independently of one another from alkyl, alkoxy, hydroxy, hydroxyalkyl or —COR^{b}, in which R^{b} is alkyl, alkoxy or NR^{c}R^{d}, where R^{c} and R^{d}, independently of one another, are hydrogen, alkyl, cycloalkyl or aryl,
c) bicycloalkyl, where these radicals may be substituted by 1, 2, 3, 4 or more than 4 of the groups given under b) as substituents of cycloalkyl,
d) a 4- to 8-membered, saturated heterocycle which, depending on the ring size, can have 1, 2, 3, 4 or more than 4 heteroatoms or groups containing heteroatoms which are preferably chosen from O, S and NR^{a}, where R^{a} is hydrogen, alkyl, acyl, cycloalkyl, heterocycloalkyl, aryl or hetaryl, and which can, if appropriate, have 1, 2, 3, 4 or more than 4 substituents which are preferably chosen independently of one another from alkyl, alkoxy, hydroxy, hydroxyalkyl, amino, aminoalkyl;
secondary amines of the formula 1.b

R²—NH—R³ (1.b)
in which
R² and R³, independently of one another, can have the meanings given for R¹, or
R² and R³, together with the nitrogen atom to which they are bonded, are a 4 to 8-membered, saturated or unsaturated heterocycle which can have 1, 2, 3 or 4 heteroatoms or groups containing heteroatoms which are preferably chosen from O, S and NR^{a}, where R^{a} is hydrogen, alkyl, acyl, cycloalkyl, heterocycloalkyl, aryl or hetaryl, and which, if appropriate, can have 1, 2, 3, 4 or more than 4 substituents which are preferably chosen independently of one another from alkyl, alkoxy, hydroxy, hydroxyalkyl, amino, aminoalkyl;
tertiary amines of the formula 1.c

NR⁵R⁶R⁷ (1.c)
in which
R⁵, R⁶ and R⁷, independently of one another, can have the meanings given for R¹, or
R⁵ and R⁶, together with the nitrogen atom to which they are bonded, are a 4- to 8-membered, saturated or unsaturated heterocycle which can have 1, 2, 3 or 4 heteroatoms or groups containing heteroatoms which are preferably chosen from O, S and NR^{a}, where R^{a} is alkyl, acyl, cycloalkyl, heterocyclo-alkyl, aryl or hetaryl, and which can optionally have 1, 2, 3, 4 or more than 4 substituents, which are preferably chosen independently of one another from alkyl, alkoxy, hydroxy, hydroxyalkyl, amino, aminoalkyl;
diamines of the formula 1.d

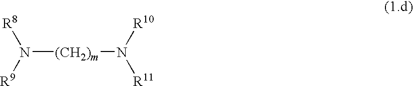

in which
m is an integer from 1 to 8,
R⁸, R⁹, R¹⁰ and R¹¹, independently of one another, are hydrogen or have the meanings given for R¹, or
R⁸ and R⁹, together with the nitrogen atom to which they are bonded, are a 4- to 8-membered, saturated or unsaturated heterocycle which can have 1, 2, 3 or 4 heteroatoms or groups containing heteroatoms which are preferably chosen from O, S and NR^{a}, where R^{a} is hydrogen, alkyl, acyl, cycloalkyl, heterocycloalkyl, aryl or hetaryl, and which can, if appropriate, have 1, 2, 3, 4 or more than 4 substituents which are preferably chosen independently of one another from alkyl, alkoxy, hydroxy, hydroxyalkyl, amino, aminoalkyl;
polyamines of the formula 1.e

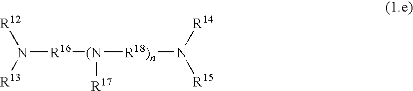

in which
m is an integer from 1 to 100, preferably 1 to 10,
R¹², R¹³, R¹⁴ and R¹⁵, independently of one another, are hydrogen or the meanings given for R¹,
R¹⁶ and R¹⁸ are a C₂- to C₆-alkylene radical, where when n>1, the radicals R¹⁸ are chosen independently of one another from C₂- to C₈-alkylene radicals,
R¹⁷ is alkyl, acyl, cycloalkyl, aryl or arylalkyl;
diaminopolyethers of the formula 1.f

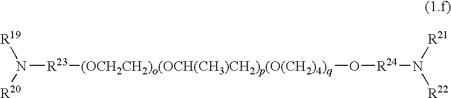

in which
the order of the alkylene oxide units is arbitrary,
o, p and q, independently of one another, are an integer from 0 to 100, where the sum of o, p and q is >0,
R¹⁹, R²⁰, R²¹ and R²², independently of one another, are hydrogen or the meanings given for R¹,
R²³ and R²⁴ are a C₂- to C₆-alkylene radical, where when n>1, the radicals R¹⁸ are chosen independently of one another from C₂- to C₆-alkylene radicals;
alkoxylated amines of the formula 1.g

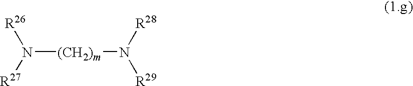

in which
m is an integer from 1 to 8,
R²⁶, R²⁷, R²⁸ and R²⁹, independently of one another, are hydrogen or the meanings given for R¹, where 1, 2, 3 or 4 of the radicals R²⁶, R²⁷, R²⁸ and R²⁹ are a group of the formula —(OCH₂CH₂)ᵣ(OCH(CH₃)CH₂)ₛ(O(CH₂)₄)ₜ—, in which the order of the alkylene oxide units is arbitrary, and r, s and t, independently of one another, are an integer from 0 to 100, where the sum of r, s and t is >0.

Preferably, at least one of the amines is a primary amine of the formula 1.a) in which R¹ is alkyl or cycloalkyl. Alkyl is preferably C₆- to C₃₀-alkyl, particularly preferably C₈- to C₁₈-alkyl. Suitable amines are then, for example, 1-hexylamine, 1-heptylamine, 1-octylamine, 1-nonylamine, 1-decylamine, 1-undecylamine, 1-undec-10-enylamine, 1-tridecylamine, 1-tetradecylamine, 1-pentadecylamine, 1-hexadecylamine, 1-heptadecylamine, 1-octadecylamine, 1-octadeca-9,12-dienylamine, 1-nonadecylamine, 1-eicosylamine, 1-eicos-9-enylamine, 1-heneicosylamine, 1-docosylamine and in particular 1-dodecylamine or amine mixtures produced from naturally occurring fatty acids, such as, for example, tallow fatty amines which predominantly comprise saturated and unsaturated C₁₄-, C₁₆- and C₁₈-alkylamines, or cocoamines which comprise saturated, mono- and diunsaturated C₆-C₂₂-, preferably C₁₂-C₁₄-alkylamines. Suitable amine mixtures are, for example, various Armeen® grades from AKZO Chemie or Noram® grades from Ceca. Suitable primary amines of the formula 1.a) in which R¹ is cycloalkyl are, for example, cyclopentylamine and cyclohexylamine.

Preference is also given to amines of the formula 1.a) in which R¹ is cycloalkylalkyl or arylalkyl. These include, for example, cyclopentylmethylamine, cyclohexylmethylamine or benzylamine.

Preference is also given to amines of the formula 1.a) in which R¹ is an alkoxycarbonylalkyl radical. These include, for example, ethyl, propyl, butyl, tert-butyl alaninate, ethyl, propyl, butyl, tert-butyl valinate, ethyl, propyl, butyl, tert-butyl leucinate, ethyl, propyl, butyl glycinate, and in particular tert-butyl glycinate.

Preference is also given to amines of the formula 1.a) in which R¹ is an alkylaminocarbonylalkyl radical. These include, for example, the ethyl-, propyl-, butyl- or tert-butylcarboxamides of the amino acids glycine, alanine, valine, leucine or isoleucine.

Preference is also given to amines of the formula 1.b) in which R² and R³, independently of one another, are alkyl and cycloalkyl.

Preference is also given to amines of the formula 1.b) in which R² and R³, together with the nitrogen atom to which they are bonded, are a 5- to 7-membered, saturated heterocycle which can have a further heteroatom or a heteroatom-containing group, which are preferably chosen from O, S and NR^{a}, where R^{a} is hydrogen, alkyl, acyl, cycloalkyl, heterocycloalkyl, aryl or hetaryl, and which can, if appropriate, have 1, 2, 3, 4 or more than 4 substituents which are preferably chosen independently of one another from alkyl, alkoxy, hydroxy, hydroxyalkyl, amino, aminoalkyl. Suitable cyclic amines of the formula 1.b) which have at least one secondary amino function are, for example, pyrrolidine, imidazolidine, N-alkylimidazolidines, N-acylimidazolidines, piperidine, 4-methylpiperidine, 3-piperidinol, 4-piperidinol, morpholine, 2,6-dimethylmorpholine, oxazolidine, piperazine, 1-alkylpiperazine, such as 1-methylpiperazine and 1-ethylpiperazine, N-(2-hydroxyethyl)piperazine, N-acylpiperazines, etc.

Suitable tertiary amines of the formula 1.c) are, for example, N,N-dimethyl-1-hexylamine, N,N-diethylhexylamine etc. In particular, 1.c) is dialkylated fatty amines, e.g. dialkylated tallow fatty amines, dialkylated hydrogenated tallow fatty amines and dialkylated cocoamines, such as N,N-dimethyl-C₁₂-C₁₄-alkylamines. Suitable amines are, for example, Noram® DM from Ceca.

Suitable cyclic amines of the formula 1.c) which have only tertiary amino functions are, for example, N-alkylpyrrolidines, such as N-methylpyrrolidine, N-alkylpiperidines, such as N-methylpiperidine, N,N′-dialkylpiperazines, such as N,N′-dimethylpiperazine, etc.

Suitable diamines of the formula 1.d) are, for example, ethylenediamine, propylenediamine, etc. Also suitable are diamines 1.d) which have at least one longer-chain C₈-C₃₀-alkyl radical. The compounds 1.d) are then, for example, N-oleyl-1,3-diaminopropane, N-dodecyl-1,3-diaminopropane or N-alkylated 1,3-diaminopropanes with alkyl radicals derived from tallow fatty acids or coconut fatty acids. Suitable amines are, for example, Dinoram® grades from Ceca, Duomeen® grades from Akzo and types 6540, 6560, 6570 and 6572 from Fina.

Suitable polyamines of the formula 1.e) are diethylenetriamine, N-methyldiethylenetriamine, N-ethyldiethylenetriamine, N,N,N′,N′-tetramethyldiethylenetriamine, N,N,N′,N′-tetraethyldiethylenetriamine, dipropylenetriamine, N-methyldipropylenetriamine, N-ethyldipropylenetriamine, N,N′-bis(3-aminopropyl)butane-1,4-diamine, triethylenetetramine, tetraethylenepentamine and mixtures thereof.

Suitable compounds of the formula 1.f) are α,ω-diaminopolyethers, which can be produced through amination of polyalkylene oxides with ammonia. Polyethers of this type and preparation methods are known to the person skilled in the art.

Suitable alkoxylated amines of the formula 1.g) are, for example, the Dinoramox® grades from Ceca and, in particular, Dinoramox® S3 or S7, i.e. ethoxylated n-tallow propylenediamines having 3 or 7 ethylene oxide units. Also suitable are hydroxyethylated or ethoxylated oleylpropylenediamines having 3 to 7, in particular having 5, ethylene oxide units.

The organic base particularly preferably comprises at least one hydroxyl-group-containing amine. Suitable hydroxyl-group-containing amines are chosen from alkanolamines, such as 2-amino-2-methylpropanol, ethanolamine, n-propanolamine, iso-propanolamine, n-butanolamine, pentanolamine, hexanolamine, heptanolamine or octanolamine, N-alkylalkanolamines, such as N-methylethanolamine, N-methylisopropanolamine, N-ethylethanolamine, N-ethylisopropanolamine, N,N-dialkylalkanolamines, such as N,N-dimethylethanolamine, N,N-dimethylpropanolamine, N,N-diethylethanolamine, dialkanolamines, such as diethanolamine, di-n-propanolamine or 2-amino-2-methyl-1,3-propanediol, N-alkyldialkanolamines, such as N-methyl-diethanolamine and preferably C₈-C₁₈-alkyldialkanolamines, trialkanolamines, such as triethanolamine or tri-n-propanolamine, and mixtures thereof. In the C₈-C₁₈-alkyl-dialkanolamines, the alkyl radicals are preferably chosen from n-octyl, n-nonyl, n-decyl, n-undecyl, n-dodecyl, n-tridecyl, n-tetradecyl, n-pentadecyl, n-hexadecyl, n-heptadecyl and n-octadecyl. These include n-octyldiethanolamine, n-nonyldiethanolamine, n-decyldiethanolamine, n-undecyldiethanolamine, n-dodecyldiethanolamine, n-tridecyl-diethanolamine, n-tetradecyldiethanolamine, n-pentadecyldiethanolamine, n-hexa-decyldiethanolamine, n-heptadecyldiethanolamine or n-octadecyldiethanolamine.

The organic base particularly preferably comprises at least one hydroxyl-group-containing amine which is chosen from monoalkanolamines, N,N-dialkylethanolamines, N-alkyldiethanolamines, triethanolamine and mixtures thereof.

In particular, the organic base comprises at least one hydroxyl-group-containing amine which is chosen from 2-amino-2-methylpropanol, N-methylethanolamine, N,N-dimethylethanolamine, N-methyldiethanolamine, triethanolamine, C₈-C₁₈-alkyldiethanolamines and mixtures thereof.

Preferably, the inorganic base is chosen from alkali metal hydroxides, particularly preferably from NaOH, KOH and mixtures thereof. In one specific embodiment, the inorganic base used is KOH.

In one preferred embodiment, the inorganic base used is KOH and the organic base used is 2-amino-2-methylpropanol.

In a further preferred embodiment, the inorganic base used is KOH and the organic base used is triethanolamine.

In a further preferred embodiment, the inorganic base used is KOH and the organic base used is at least one C₈-C₁₈-alkyldiethanolamine.

Preferably, at least 40%, particularly preferably at least 50%, in particular at least 70%, especially at least 80%, of the acid groups of the polymer component are neutralized with at least one inorganic base (based on free acid groups, i.e. anionogenic groups, of the polymer component used for the neutralization).

Preferably, at least 1%, particularly preferably at least 3%, especially at least 5%, of the acid groups of the polymer component are neutralized with at least one organic base.

The polymer component A) comprises at least one polyurethane which is chosen from linear, branched and crosslinked polyurethanes. In one preferred embodiment, the component A) comprises at least one water-soluble or water-dispersible, carboxylic-acid-group-containing, if appropriate crosslinked polyurethane.

Suitable carboxylic-acid-group-containing polyurethanes preferably have an acid number in the range from 12 to 150, particularly preferably 30 to 90. Preferably, the polyurethanes have at least one glass transition temperature T_{G}, which is at least 15° C. This T_{G} can, for example, have values up to 120° C. and is preferably in a range from 30 to 90° C.

Preferably, the component A) comprises at least one polyurethane which comprises, in incorporated form,
a) at least one polymer with a number-average molecular weight of more than 280 which has at least two groups reactive toward isocyanate groups per molecule,
b) at least one compound which has two active hydrogen atoms and at least one anionogenic or anionic group per molecule,
c) if appropriate at least one compound which has two active hydrogen atoms and at least one cationogenic or cationic group per molecule,
d) if appropriate at least one compound with a molecular weight in the range from 56 to 280 g/mol which comprises at least two groups reactive toward isocyanate groups per molecule,
e) at least one polyisocyanate.

### Component a)

Component a) is preferably a polymer with a number-average molecular weight in the range from about 300 to 50 000, particularly preferably about 400 to 40 000, in particular 500 to 30 000. Polymers a) which can be used are, for example, polyesterdiols, polycarbonatediols, polyetherols, polysiloxanes, polymers based on α,β-ethylenically unsaturated monomers and mixtures thereof.

Preferred polyetherols a) are polyalkylene glycols, e.g. polyethylene glycols, polypropylene glycols, polytetrahydrofurans, etc., copolymers of ethylene oxide and propylene oxide or block copolymers of ethylene oxide, propylene oxide and/or butylene oxide which comprise the copolymerized alkylene oxide units in random distribution or in the form of blocks. Suitable polytetrahydrofurans a) can be produced by cationic polymerization of tetrahydrofuran in the presence of acidic catalysts, such as, for example, sulfuric acid or fluorosulfuric acid. Such preparation methods are known to the person skilled in the art. Suitable compounds a) are also α,ω-diaminopolyethers, which can be produced by amination of polyalkylene oxides with ammonia.

As component a), preference is given to using polytetrahydrofurans and mixtures which comprise these. The component a) is then preferably chosen from polytetrahydrofurans of the general formula

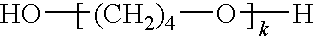

where k=4 to 40, preferably 6 to 35, and mixtures thereof.

These polytetrahydrofurans preferably have a number-average molecular weight in the range from 650 to 2000, preferably 750 to 1800, in particular 800 to 1500.

Suitable polyesterdiols a) preferably have a number-average molecular weight in the range from about 400 to 5000, preferably 500 to 3000, in particular 600 to 2000.

Suitable polyesterdiols a) are all those which are usually used for producing polyurethanes, in particular those based on aromatic dicarboxylic acids, such as terephthalic acid, isophthalic acid, phthalic acid, Na or K sulfoisophthalic acid etc., aliphatic dicarboxylic acids, such as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane.

Preference is given to polyesterdiols a) based on aromatic and aliphatic dicarboxylic acids and aliphatic diols, in particular those in which the aromatic dicarboxylic acid constitutes 10 to 95 mol %, in particular 40 to 90 mol %, of the total dicarboxylic acid fraction (remainder aliphatic dicarboxylic acids).

Particularly preferred polyesterdiols a) are the reaction products of phthalic acid/diethylene glycol, isophthalic acid/1,4-butanediol, isophthalic acid/adipic acid/1,6-hexanediol, 5-NaSO₃-isophthalic acid/phthalic acid/adipic acid/1,6-hexanediol, adipic acid/ethylene glycol, isophthalic acid/adipic acid/neopentyl glycol, isophthalic acid/adipic acid/neopentyl glycol/diethylene glycol/dimethylolcyclohexane and 5-NaSO₃-isophthalic acid/isophthalic acid/adipic acid/neopentyl glycol/diethylene glycol/dimethanolcyclohexane, isophthalic acid/adipic acid, neopentyl glycol/dimethylolcyclohexane.

As component a), preference is also given to polyesterdiols based on linear or branched, C₈-C₃₀-di- or polycarboxylic acids and C₈-C₃₀-hydroxycarboxylic acids. Preferred carboxylic acids and hydroxycarboxylic acids are, for example, azelaic acid, dodecanedioic acid, suberic acid, pimelic acid, sebacic acid, tetradecanedioic acid, citric acid, ricinoleic acid, hydroxystearic acid and mixtures thereof. Preferably, 1,4-butanediol, 1,5-pentanediol, 1,6-hexanediol, neopentyl glycol, 1,4-dimethylolcyclohexane, diethylene glycol and mixtures thereof are used as diol component for producing these polyesterdiols.

Also suitable as component a) are polycarbonatediols. Polycarbonatediols are formed, for example, by condensation of phosgene or carbonic esters, such as diphenyl carbonate or dimethyl carbonate, with dihydroxy compounds. Suitable dihydroxy compounds are aliphatic or aromatic dihydroxy compounds. Suitable aliphatic dihydroxy compounds are those mentioned above with the polyesterdiols. Aromatic dihydroxy compounds which may be mentioned are, for example, bisphenols, such as 2,2-bis(4-hydroxyphenyl)propane (bisphenol A), tetraalkylbisphenol A, 4,4-(meta-phenylenediisopropyl)diphenol (bisphenol M), 4,4-(para-phenylenediisopropyl)diphenol, 1,1-bis(4-hydroxyphenyl)-3,3,5-trimethylcyclohexane (BP-TMC), 2,2-bis(4-hydroxy-phenyl)-2-phenylethane, 1,1-bis(4-hydroxyphenyl)cyclohexane (bisphenol Z), and, if appropriate, mixtures thereof. The polycarbonates can be branched by using small amounts of branching agents. Suitable branching agents include phloroglucine, 4,6 dimethyl-2,4,6-tri(4-hydroxyphenyl)heptene-2,4,6-dimethyl-2,4,6-tri(4-hydroxyphenyl)heptane; 1,3,5-tri(4-hydroxyphenyl)benzene; 1,1,1-tri(4-hydroxyphenyl)heptane; 1,3,5-tri(4-hydroxyphenyl)benzene; 1,1,1-tri(4-hydroxyphenyl)ethane; tri(4-hydroxyphenyl)phenylmethane, 2,2-bis[4,4-bis(4-hydroxyphenyl)cyclohexyl]propane; 2,4-bis(4-hydroxyphenylisopropyl)phenol; 2,6 bis(2-hydroxy-5′-methylbenzyl)-4-methylphenol; 2-(4-hydroxyphenol)-2-(2,4-dihydroxyphenyl)propane; hexa(4-(4-hydroxyphenylisopropyl)phenyl)orthoterephthalic esters; tetra(4-hydroxyphenol)methane; tetra(4-(4-hydroxyphenylisopropyl)phenoxy)methane; a,a′,a″-tris(4-hydroxyphenyl)-1,3,5-triisopropylbenzene; 2,4-dihydroxybenzoic acid; trimesic acid; cyanuric chloride; 3,3-bis(3-methyl-4-hydroxy-phenyl)-2-oxo-2,3-dihydroindole, 1,4-bis(4′,4″-dihydroxytriphenyl)methyl)benzene and, in particular, 1,1,1-tri(4-hydroxyphenyl)ethane and bis(3-methyl-4-hydroxyphenyl)-2-oxo-2,3-dihydroindole.

Suitable polysiloxanes a) preferably have a number-average molecular weight in the range from about 300 to 50 000, particularly preferably 400 to 30 000.

Preferred compounds of component a) are polysiloxanes of the general formula I.1

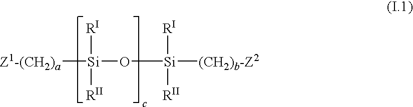

in which
a and b, independently of one another, are 1 to 8,
c is 2 to 1000,
R^{I} and R^{II}, independently of one another, are alkyl, cycloalkyl or aryl,
Z¹ and Z², independently of one another, are radicals of the formula II

—(OCH₂CH₂)ᵤ(OCH(CH₃)CH₂)ᵥ(O(CH₂)₄)_{w}—X¹—H (II)

where
in the formula II, the order of the alkylene oxide units is arbitrary,
u, v and w, independently of one another, are an integer from 0 to 500, where the sum of u, v and w is >0,
X¹ is O or NR^{III}, in which R^{III} is hydrogen, alkyl, cycloalkyl or aryl.

Preferably, in the formula I.1, the sum of u, v and w is chosen so that the molecular weight of the polysiloxanes a) is in a range from about 300 to 30 000.

Preferably, the total number of alkylene oxide units in the polysiloxanes a), i.e. the sum of u, v and w, is in a range from about 3 to 200, preferably 5 to 180.

Preferably, in the compounds of the formula I, the radicals R^{I} and R^{II}, independently of one another, are chosen from methyl, ethyl, cyclohexyl, phenyl and benzyl. R^{I} and R^{II} are both particularly preferably methyl.

One example of suitable compounds of the formula I.1 are the bis(polyethylene glycol)dimethicones of the general formula I.1a

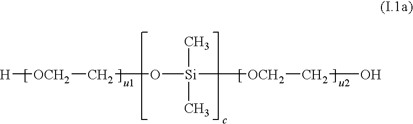

in which
c is an integer from 3 to 500, preferably 5 to 250, and
u1 and u2, independently of one another, are 2 to 500, in particular 3 to 250, specifically 5 to 100.

Preferably, the compounds of component a) are also chosen from polysiloxanes of the general formula I.2

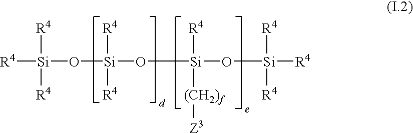

in which
the order of the siloxane units is arbitrary,
the radicals R⁴ are in each case independently of one another alkyl, cycloalkyl or aryl,
d is an integer from 2 to 1000,
e is an integer from 2 to 100,
f is an integer from 2 to 8, and
Z³ is a radical of the formula II, as defined above,
and mixtures thereof.

One example of suitable compounds of the formula I.2 are the ethoxylated and/or propoxylated polydimethylsiloxanes of the general formula I.2a

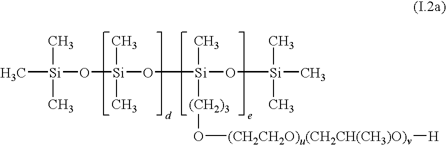

in which
the order of the siloxane units is arbitrary,
d is an integer from 2 to 1000, preferably 3 to 500, in particular 5 to 100,
e is an integer from 2 to 100, preferably 3 to 50, in particular 4 to 20, and
u and v, independently of one another, are an integer from 0 to 500, preferably 0 to 250, where the sum of u and v is ≧1, preferably ≧5, in particular ≧10.
Suitable compounds of the formula I.1 are available under the name Wacker-Belsil® DMC 6031 and Pluriol® ST 4005 (BASF Aktiengesellschaft).

Suitable compounds of component a) are quite general siloxane derivatives which have at least two polyether chains with terminal OH groups. These include the compounds known under the INCI names dimethicone copolyols or silicone surfactants, as are available under the trade names Abil®, Degussa-Goldschmidt, Alkasil® (Rhone Poulenc), Silicone Polyol Copolyol® (Genesee Polymers Corporation), Belsil® (Wacker), Silwet® (OSI), Dow Corning (Dow Corning) or Tecopren (Degussa-Goldschmidt).

Also suitable as component a) are polymers based on α,β-ethylenically unsaturated monomers which have at least two groups reactive toward NCO groups. The groups reactive toward NCO groups are preferably chosen from hydroxyl groups, primary and secondary amino groups. Such polymers are also referred to below for short as poly-acrylates. Compounds which can be derived from acrylic acid or methacrylic acid are sometimes referred to below in shortened form by adding the syllable “(meth)” to the compound derived from acrylic acid.

To prepare the polyacrylates a), preference is given to using at least one α,β-ethylenically unsaturated monomer which has at least one functional group which is chosen from hydroxyl groups, primary or secondary amine groups or groups which can be converted into such amine groups. Preferably, this monomer is chosen from esters of α,β-ethylenically unsaturated mono- and dicarboxylic acids with amino alcohols which have at least one primary or secondary amino group, amides of α,β-ethylenically unsaturated mono- and dicarboxylic acids with diamines which have at least one primary or secondary amino group, esters of α,β-ethylenically unsaturated mono- and dicarboxylic acids with diols, amides of α,β-ethylenically unsaturated mono- and dicarboxylic acids with amino alcohols which have a primary or secondary amino group, N,N-diallylamine and mixtures thereof (=monomer α)).

The polyacrylates comprise preferably 0.1 to 20% by weight, particularly preferably 0.3 to 10% by weight, in particular 0.5 to 7% by weight, specifically at most 4% by weight, based on the total weight of the monomers used for the polymerization, of at least one monomer α) in copolymerized form.

Suitable compounds α) are, for example, the esters of α,β-ethylenically unsaturated mono- and dicarboxylic acids with amino alcohols, preferably C₂-C₁₂-amino alcohols. These can preferably be C₁-C₈-monoalkylated on the amine nitrogen. Suitable as acid component of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof. Preference is given to N-methylaminoethyl(meth)acrylate, N-ethylaminoethyl(meth)acrylate, N-(n-propyl)aminoethyl(meth)acrylate, N-(n-butyl)aminoethyl(meth)acrylate, tert-butylaminoethyl(meth)acrylate. Particular preference is given to N-tert-butylaminoethyl methacrylate.

Suitable monomers α) are also the amides of the abovementioned α,β-ethylenically unsaturated mono- and dicarboxylic acids with diamines which have at least one primary or secondary amino group.

Suitable monomers α) are, for example, N-methylaminoethyl(meth)acrylamide, N-ethylaminoethyl(meth)acrylamide, N-(n-propyl)aminoethyl(meth)acrylamide, N-(n-butyl)aminoethyl(meth)acrylamide and N-tert-butylaminoethyl(meth)acrylamide.

Suitable monomers a) are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Further suitable monomers α) are 2-hydroxyethylacrylamide, 2-hydroxyethyl-methacrylamide, 2-hydroxyethylethacrylamide, 2-hydroxypropylacrylamide, 2-hydroxy-propylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexyl-methacrylamide, 3-hydroxy-2-ethylhexylacrylamide and 3-hydroxy-2-ethylhexyl-methacrylamide.

A preferred monomer α) is also N,N-diallylamine.

Preferred monomers α) are tert-butylaminoethyl methacrylate, diallylamine and mixtures thereof.

The polyacrylates a) containing amine groups and/or hydroxyl groups can additionally comprise, in copolymerized form, at least one further nonionic, amide-group-containing, α,β-ethylenically unsaturated monomer different from the abovementioned monomers α) and copolymerizable therewith (=monomer β)). These are preferably water-soluble monomers.

Preferably, the polyacrylates a) comprise 0 to 80% by weight, particularly preferably 1 to 75% by weight, in particular 10 to 70% by weight, based on the total weight of the monomers used for the polymerization, of at least one monomer β) in copolymerized form.

Preferably, this component β) is chosen from N-vinyllactams, N-vinylamides of saturated C₁-C₈-monocarboxylic acids, primary amides of α,β-ethylenically unsaturated monocarboxylic acids and N-alkyl and N,N-dialkyl derivatives thereof, and mixtures thereof.

Preferably, the polyacrylates a) additionally comprise at least one N-vinyllactam β) in copolymerized form. Suitable monomers β) are unsubstituted N-vinyllactams and N-vinyllactam derivatives which, for example, can have one or more C₁-C₆-alkyl substituents, such as methyl, ethyl, n-propyl, isopropyl, n-butyl, sec-butyl, tert-butyl, etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc. Preference is given to using N-vinylpyrrolidone and/or N-vinylcaprolactam.

Open-chain N-vinylamide compounds suitable as monomers β) are, for example, N-vinylformamide, N-vinyl-N-methylformamide, N-vinylacetamide, N-vinyl-N-methyl-acetamide, N-vinyl-N-ethylacetamide, N-vinylpropionamide, N-vinyl-N-methyl-propionamide and N-vinylbutyramide.

Suitable monomers β) are also acrylamide and methacrylamide.

Suitable monomers β) are also N—C₁-C₈-alkyl- and N,N-di(C₁-C₈-)alkylamides of α,β-ethylenically unsaturated monocarboxylic acids. These include N-methyl(meth)acrylamide, N-ethyl(meth)acrylamide, N-propyl(meth)acrylamide, N-isopropyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide.

Suitable monomers β) are also N-(n-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-penta-decyl(meth)acrylamide, N-palmityl(meth)acrylamide, N-heptadecyl(meth)acrylamide, N-nonadecyl(meth)acrylamide, N-arrachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide.

The polyacrylates a) can additionally comprise, in copolymerized form, at least one, preferably water-soluble, monomer which is chosen from α,β-ethylenically unsaturated compounds with anionogenic and/or anionic groups (=monomer γ)). Preferably, the polyacrylates a) comprise 0 to 30% by weight, particularly preferably 0.1 to 20% by weight, in particular 0.5 to 15% by weight, based on the total weight of the monomers used for the polymerization, of at least one monomer γ) in copolymerized form.

If polyacrylates a) which comprise at least one monomer γ) in the free acid form in copolymerized form are used in the polyurethanes, then component a) can partially or completely replace component b).

Preferably, the anionogenic/anionic monomers γ) are chosen from monoethylenically unsaturated carboxylic acids, sulfonic acids, phosphonic acids and mixtures thereof.

The monomers γ) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers γ) also include the half-esters of monoethylenically unsaturated dicarboxylic acids having 4 to 10, preferably 4 to 6, carbon atoms, e.g. of maleic acid, such as monomethyl maleate. The monomers γ) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid. The monomers γ) also include the salts of the above-mentioned acids, in particular the sodium, potassium and ammonium salts, and the salts with the abovementioned amines.

The monomers γ) can be used as they are or as mixtures with one another. The stated parts by weight all refer to the free base form or the free acid form.

As monomer γ), particular preference is given to acrylic acid, methacrylic acid and mixtures thereof.

The polyacrylates a) can additionally comprise, in copolymerized form, at least one further monomer different from the amine-group-containing monomers α) which is chosen from α,β-ethylenically unsaturated compounds with cationogenic and/or cationic groups (=monomer δ)). Monomers δ are only used in amounts such that the polyurethanes used according to the invention have an excess of anionogenic/anionic groups over cationogenic/cationic groups. Preferably, the polymers comprise 0 to 30% by weight, particularly preferably 0.1 to 20% by weight, in particular 0.5 to 15% by weight, based on the total weight of the monomers used for the polymerization, of at least one monomer δ) in copolymerized form.

Preferably, the cationogenic and/or cationic groups of component δ) are nitrogen-containing groups, such as tertiary amino groups, and quaternary ammonium groups. Charged cationic groups can be produced from amine nitrogens either by protonation, e.g. with carboxylic acids, such as lactic acid, or mineral acids, such as phosphoric acid, sulfuric acid and hydrochloric acid, or by quaternization, e.g. with alkylating agents, such as C₁-C₄-alkyl halides or sulfates. Examples of such alkylating agents are ethyl chloride, ethyl bromide, methyl chloride, methyl bromide, dimethyl sulfate and diethyl sulfate.

Suitable monomers δ) are, for example, N,N-dialkylaminoalkyl(meth)acrylates, such as N,N-dimethylaminomethyl(meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate, N,N-dimethylaminocyclohexyl(meth)acrylate etc.

Also suitable as monomers δ) are N,N-dialkylaminoalkyl(meth)acrylamides, such as N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[2-(diethylamino)ethyl]methacrylamide, N-[4-(dimethylamino)cyclohexyl]acrylamide and N-[4-(dimethylamino)cyclohexyl]methacrylamide. Preference is given to N,N-dimethylaminopropyl acrylate, N,N-dimethylaminopropyl methacrylate, N-[3-dimethylamino)propyl]acrylamide and N-[3-(dimethylamino)propyl]meth-acrylamide.

Suitable monomers δ) are also vinyl and allyl-substituted nitrogen heterocycles, such as vinylimidazole, N-vinyl-2-alkylimidazoles, e.g. N-vinyl-2-methylimidazole, and 2- and 4-vinylpyridine, 2- and 4-allylpyridine, and the salts thereof.

Furthermore, suitable monomers δ) are alkylallylamines and allylammonium salts, such as diallylmethylamine and diallyldimethylammonium chloride (DADMAC).

N-Vinylimidazole is preferred as monomer δ).

The polyacrylates a) can additionally comprise, in copolymerized form, at least one further monomer ε) which is preferably chosen from esters of α,β-ethylenically unsaturated mono- and dicarboxylic acids with C₁-C₃₀-alkanols, esters of vinyl alcohol and allyl alcohol with C₁-C₃₀-monocarboxylic acids, vinyl ethers, vinylaromatics, vinyl halides, vinylidene halides, C₁-C₈-monoolefins, nonaromatic hydrocarbons with at least two conjugated double bonds and mixtures thereof.

Preferably, the polyacrylates a) comprise up to 50% by weight, particularly preferably up to 30% by weight and especially preferably up to 15% by weight, based on the total weight of the monomers used for the polymerization, of at least one monomer ε) in copolymerized form. If at least one monomer c) is used for the polymerization, then it is preferably used in an amount of at least 0.1% by weight, particularly preferably of at least 1% by weight.

Suitable monomers ε) are then methyl(meth)acrylate, methyl ethacrylate, ethyl (meth)acrylate, ethyl ethacrylate, n-butyl(meth)acrylate, tert-butyl(meth)acrylate, tert-butyl ethacrylate, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl (meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arrachinyl(meth)acrylate, behenyl(meth)acrylate, lignocerenyl(meth)acrylate, cerotinyl(meth)acrylate, melissinyl(meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate and mixtures thereof. Preferred monomers e) are the esters of α,β-ethylenically unsaturated mono- and dicarboxylic acids with C₁-C₄-alkanols.

Suitable monomers ε) are also vinyl acetate, vinyl propionate, vinyl butyrate and mixtures thereof.

Suitable monomers ε) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The abovementioned monomers ε) can be used individually or in the form of any mixtures.

The polyacrylates a) are prepared by customary methods known to the person skilled in the art, e.g. by solution polymerization, precipitation polymerization, suspension polymerization or emulsion polymerization. Preference is given to preparation by solution or precipitation polymerization.

Preferred solvents for the solution polymerization are aqueous solvents, such as water, water-miscible solvents and mixtures of water with water-miscible solvents, for example ketones, such as acetone and methyl ethyl ketone, alcohols, such as methanol, ethanol, n-propanol, isopropanol, n-butanol, sec-butanol, tert-butanol, n-hexanol and cyclo-hexanol, and glycols, such as ethylene glycol, propylene glycol and butylene glycol, and the methyl or ethyl ethers of the dihydric alcohols, diethylene glycol, triethylene glycol, polyethylene glycols with number-average molecular weights up to about 3000, glycerol and dioxane.

The precipitation polymerization takes place, for example, in an ester, such as ethyl acetate or butyl acetate, as solvent. The resulting polymer particles precipitate out of the reaction solution and can be isolated by customary methods, such as filtration by means of subatmospheric pressure. As a rule, with precipitation polymerization, polymers with higher molecular weights are obtained than with solution polymerization.

The polymerization temperatures are preferably in a range from about 30 to 120° C., particularly preferably 40 to 100° C. The polymerization usually takes place under atmospheric pressure, although it can also proceed under reduced or increased pressure. A suitable pressure range is between 1 and 5 bar.

To prepare the polymers, the monomers can be polymerized with the help of initiators forming free radicals.

Initiators for the free-radical polymerization which can be used are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis(o-tolyl)peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, 2,2′-azobis(2-amidinopropane) hydrochloride (V50 from Wako Pure Chemicals Industries, Ltd.), or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{I}.

To adjust the molecular weight, the polymerization can take place in the presence of at least one regulator. Regulators which can be used are the customary compounds known to the person skilled in the art, such as, for example, sulfur compounds, e.g. mercaptoethanol, 2-ethylhexyl thioglycolate, thioglycolic acid or dodecyl mercaptan, and tribromochloromethane or other compounds which have a regulating effect on the molecular weight of the polymers obtained. A preferred regulator is cysteine.

Further suitable as component a) are polymers with a number-average molecular weight of at least 1000 and which have a hydroxyl number of at least 0.1 g of KOH/g and/or an amine number of at least 0.1 g of KOH/g. These include preferably hydroxyl-group-containing polymers which have an OH number in the range from 0.3 to 60, particularly preferably from 0.9 to 30 and in particular from 1.5 to 21. These also include amine-group-containing polymers which have an amine number in the range from 0.3 to 60, particularly preferably from 0.9 to 30 and in particular from 1.5 to 21. These also include hydroxyl- and amine-group-containing polymers in which the sum of hydroxyl number and amine number is in a range from 0.3 to 60, preferably from 0.9 to 30 and in particular from 1.5 to 21.

### Component b)

Preferred compounds b) with two active hydrogen atoms and at least one anionogenic and/or anionic group per molecule are, for example, compounds with carboxylate, sulfonate and/or phosphate groups. As component b), 2,2-hydroxymethylalkylcarboxylic acids, such as dimethylolpropanoic acid, 2,2-dimethylolbutanoic acid and mixtures which comprise 2,2-hydroxymethylalkylcarboxylic acids, such as dimethylolpropanoic acid and/or 2,2-dimethylolbutanoic acid, are particularly preferred.

Suitable diamines and/or diols b) with anionogenic or anionic groups are compounds of the formula

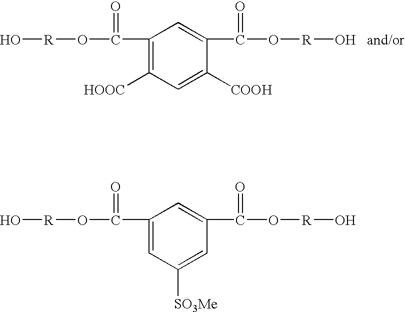

in which R is in each case a C₂-C₁₈-alkylene group and Me is Na or K.

As component b), it is also possible to use compounds of the formula

H₂N(CH₂)_{w}—NH—(CH₂)ₓ—COO⁻M⁺

H₂N(CH₂)_{w}—NH—(CH₂)ₓ—SO₃⁻M⁺

in which w and x, independently of one another, are an integer from 1 to 8, in particular 1 to 6, and M is Li, Na or K, and compounds of the formula

H₂N(CH₂CH₂O)_{y}(CH₂CH(CH₃)O)_{z}(CH₂)_{w}—NH—(CH₂)ₓ—SO₃⁻M⁺

in which w and x have the meanings given above, y and z, independently of one another, are an integer from 0 to 50, where at least one of the two variables y or z is ≧0. The order of the alkylene oxide units here is arbitrary. The last-mentioned compounds preferably have a number-average molecular weight in the range from about 400 to 3000. A suitable compound of this type is, for example, Poly ESP 520 from Raschig.

### Component c)

Preferred compounds c) are those which have two active hydrogen atoms and at least one tertiary amino group and/or quaternary ammonium group per molecule. Compounds c) which can be used are, for example, compounds of the general formulae

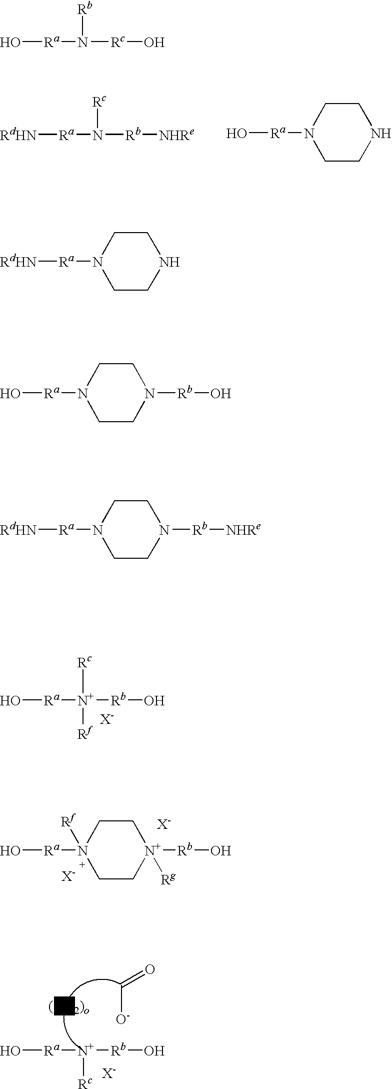

in which
R^{a} and R^{b}, which may be identical or different, are C₂-C₈-alkylene,
R^{c}, R^{f} and R^{g}, which may be identical or different, are C₁-C₆-alkyl, phenyl or phenyl-C₁-C₄-alkyl,
R^{d} and R^{e}, which may be identical or different, are H or C₁-C₆-alkyl,
o is 1, 2 or 3,
X^{e} is chloride, bromide, iodide, C₁-C₆-alkylsulfate or SO₄²⁻/₂. Particular preference is given to N—(C₁-C₆-alkyl)diethanolamines, such as methyldiethanolamine.

Preferably, the polyurethanes A) have a considerable excess of anionogenic/anionic groups over cationogenic/cationic groups. In one specific embodiment, to prepare the polyurethanes, compounds with ionogenic or ionic groups are used in amounts such that the polyurethane A) has a molar excess of anionogenic/anionic groups over cationogenic/cationic groups of more than 2:1, preferably at least 5:1.

### Component d)

Suitable compounds d) are, for example, diols, diamines, amino alcohols and mixtures thereof.

As component d), preference is given to using diols whose molecular weight is in a range from about 62 to 286 g/mol. These include, for example, diols having 2 to 18 carbon atoms, preferably 2 to 10 carbon atoms, such as 1,2-ethanediol, 1,3-propanediol, 1,4-butanediol, 1,6-hexanediol, 1,5-pentanediol, 1,10-decanediol, 2-methyl-1,3-propanediol, 2,2-dimethyl-1,3-propanediol, di-, tri-, tetra-, penta- and hexa-ethylene glycol, neopentyl glycol, cyclohexanedimethylol and mixtures thereof. Particular preference is given to neopentyl glycol.

Preferred amino alcohols d) are, for example, 2-aminoethanol, 2-(N-methyl-amino)ethanol, 3-aminopropanol, 4-aminobutanol, 1-ethylaminobutan-2-ol, 2-amino-2-methyl-1-propanol, 4-methyl-4-aminopentan-2-ol etc.

Preferred diamines d) are, for example, ethylenediamine, propylenediamine, 1,4-diaminobutane, 1,5-diaminopentane and 1,6-diaminohexane.

The compounds specified as component d) can be used individually or in mixtures. Particular preference is given to using 1,2-ethanediol, 1,4-butanediol, 1,6-hexanediol, neopentyl glycol, diethylene glycol, cyclohexanedimethylol and mixtures thereof.

In one preferred embodiment, the polyurethane used according to the invention is a crosslinked polyurethane. These generally comprise, in incorporated form, at least one compound d) which comprises more than two active hydrogen atoms per molecule. Suitable as component d) are compounds which have more than two groups reactive toward NCO groups, which are preferably chosen from hydroxyl groups, primary and secondary amine groups.

These compounds of component d) are preferably chosen from triols, polyols with more than three hydroxyl groups, triamines, polyamines with more than three primary or secondary amino groups, amine- and/or hydroxyl-group-containing polymers and mixtures thereof.

Preferably suitable as component d) are mixtures which have compounds with two and compounds with three groups reactive toward NCO groups, and whose molecular weight is in the range from 80 to 280 g/mol.

Preferably, the compounds of component d) are chosen from triols and polyvalent polyols. Preferred triols are, for example, glycerol and trimethylolpropane. Preferred triols d) are also the triesters of hydroxycarboxylic acids with trihydric alcohols. These are preferably triglycerides of hydroxycarboxylic acids, such as, for example, lactic acid, hydroxystearic acid and ricinoleic acid. Also suitable are naturally occurring mixtures which comprise hydroxycarboxylic acid triglycerides, in particular ricinus oil. Preferred polyvalent polyols c) are, for example, erythritol, pentaerythritol and sorbitol.

Preferred triamines d) are, for example, diethylenetriamine, N,N′-diethyldiethylene-triamine etc. Preferred polyvalent polyamines are, for example, triethylenetetramine etc. and α,ω-diaminopolyethers, which can be prepared by amination of polyalkylene oxides with ammonia.

### Polyisocyanate e)

Suitable polyisocyanates e) are chosen from compounds with 2 to 5 isocyanate groups, isocyanate prepolymers with an average number of from 2 to 5 isocyanate groups, and mixtures thereof. These include, for example, aliphatic, cycloaliphatic and aromatic di-, tri- and polyisocyanates. Suitable diisocyanates e) are, for example, tetramethylene diisocyanate, hexamethylene diisocyanate (HDI), 2,3,3-trimethylhexamethylene diisocyanate, 1,4-cyclohexylene diisocyanate, isophorone diisocyanate (IPDI), 1,4-phenylene diisocyanate, 2,4- and 2,6-tolylene diisocyanate and isomer mixtures thereof (e.g. 80% 2,4 isomer and 20% 2,6 isomer), 1,5-naphthylene diisocyanate, 2,4 and 4,4′-diphenylmethane diisocyanate (MDI), and 4,4′-dicyclohexylmethane diisocyanate (HMDI). A suitable triisocyanate is, for example, triphenylmethane 4,4′,4″-triisocyanate. Also suitable are isocyanate prepolymers and polyisocyanates which are obtainable by addition of the abovementioned isocyanates onto polyfunctional hydroxyl- or amine-group-containing compounds. Also suitable are polyisocyanates which form through biuret, allophanate or isocyanurate formation.

Preferably, component e) is chosen from HDI, IPDI, MDI, HMDI and mixtures thereof. Preferably, component e) comprises at least one diisocyanate with two differently reactive isocyanate groups. The component particularly preferably comprises isophorone diisocyanate and its biurets, allophanates and/or isocyanurates. In one specific embodiment, component e) comprises isophorone diisocyanate and hexamethylene diisocyanate or consists of a mixture of isophorone diisocyanate and hexamethylene diisocyanate.

The polyurethanes used in the compositions according to the invention are prepared by reacting the compounds of components a), b) and e), and, if appropriate, c) and/or d). The temperature here is in a range from about 30 to 140° C., preferably about 40 to 100° C. The reaction can take place without solvents or in a suitable inert solvent or solvent mixture. Suitable solvents are aprotic polar solvents, e.g. tetrahydrofuran, ethyl acetate, N-methylpyrrolidone, dimethylformamide and preferably ketones, such as acetone and methyl ethyl ketone. The reaction preferably takes place under an inert-gas atmosphere, such as, for example, under nitrogen. The components are used in amounts such that the ratio of NCO equivalent of the compounds of component e) to equivalent of active hydrogen atom of components a), b), c), d) is in a range from about 0.8:1 to 1.25:1, preferably 0.85:1 to 1.2:1, in particular 1.05:1 to 1.15:1. If the resulting polyurethanes still have free isocyanate groups, then these are finally deactivated by adding bases. Suitable bases are those used according to the invention as neutralizing agents.

If, in the preparation of the polyurethanes, a water-miscible organic solvent is used, then this can be removed afterwards by customary methods known to the person skilled in the art, e.g. by distillation at reduced pressure. Prior to separating off the solvent, water can additionally be added to the polyurethane. Replacement of the solvent with water gives a solution or dispersion of the polymer, from which, if desired, the polymer can be obtained in the usual way, e.g. through spray-drying.

Instead of or in addition to at least one acid-group-containing polyurethane, component A) can comprise at least one further acid-group-containing polymer S). Suitable acid-group-containing polymers S) are obtainable, for example, by free-radical polymerization of α,β-ethylenically unsaturated monomers. Use is made here of monomers S.1) which comprise at least one free-radically polymerizable, α,β-ethylenically unsaturated double bond and at least one anionogenic and/or anionic group per molecule.

Preferably, the monomers S.1) are chosen from monoethylenically unsaturated carboxylic acids, sulfonic acids, phosphonic acids and mixtures thereof.

The monomers S.1) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers S.1) also include the half-esters of monoethylenically unsaturated dicarboxylic acids having 4 to 10, preferably 4 to 6, carbon atoms, e.g. of maleic acid, such as monomethyl maleate. The monomers S.1) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropane-sulfonic acid, vinylphosphonic acid and allylphosphonic acid. The monomers S.1) also include the salts of the abovementioned acids, in particular the sodium, potassium and ammonium salts, and the salts with the abovementioned amines. The monomers S.1) can be used as they are or as mixtures with one another. The stated parts by weight all refer to the acid form.

Component S.1) is preferably chosen from acrylic acid, methacrylic acid and mixtures thereof.

Suitable comonomers for producing the polymers S) are in principle all α,β-ethylenically unsaturated compounds copolymerizable with the monomers S.1).

In one specific embodiment, the polymers S) comprise at least one crosslinking monomer S.2) in copolymerized form. Suitable crosslinkers S.2) are compounds with two or more than two ethylenically unsaturated, nonconjugated double bonds. Preference is given to using crosslinkers S.2) in an amount of from 0.01 to 3% by weight, particularly preferably 0.1 to 2% by weight, based on the total weight of the monomers used for the polymerization of the polymers S).

Suitable crosslinkers S.2) are, for example, acrylic esters, methacrylic esters, allyl ethers or vinyl ethers of at least dihydric alcohols. The OH groups of the parent alcohols here may be completely or partially etherified or esterified; however, the crosslinkers comprise at least two ethylenically unsaturated groups.

Further suitable crosslinkers S.2) are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. Further suitable crosslinkers S.2) are esters of unsaturated carboxylic acids with polyhydric alcohols. Furthermore, suitable crosslinkers S.2) are straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes having molecular weights of from 200 to 20 000. Further suitable crosslinkers S.2) are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylene-triamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above. Also suitable are triallylamine and triallylmonoalkylammonium salts, e.g. triallylmethylammonium chloride or methyl sulfate, as crosslinker S.2).

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea. Further suitable crosslinkers S.2) are divinyldioxane, tetraallylsilane or tetravinylsilane. Particularly preferably used crosslinkers S.2) are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or poly-hydric alcohols which have been reacted with ethylene oxide and/or propylene oxide and/or epichlorohydrin.

Furthermore, the polymers S) preferably comprise at least one monomer S.3) in copolymerized form which is chosen from compounds of the general formula III)

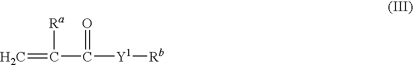

in which
R^{a} is hydrogen or C₁-C₈-alkyl,

### Y¹ is O, NH or NR^{c}, and

R^{b} and R^{c}, independently of one another, are C₁-C₃₀-alkyl or C₅-C₈-cycloalkyl, where the alkyl groups may be interrupted by up to four nonadjacent heteroatoms or heteroatom-containing groups which are chosen from O, S and NH.

Preferably, R^{a} in the formula III is hydrogen or C₁-C₄-alkyl, in particular hydrogen, methyl or ethyl.

Preferably, R^{b} in the formula III is C₁-C₈-alkyl, preferably methyl, ethyl, n-butyl, isobutyl, tert-butyl or a group of the formula —CH₂CH₂—NH—C(CH₃)₃.

If R^{c} is alkyl, then it is preferably C₁-C₄-alkyl, such as methyl, ethyl, n-propyl, n-butyl, isobutyl and tert-butyl.

Suitable monomers S.3) are methyl(meth)acrylate, methyl ethacrylate, ethyl(meth)acrylate, ethyl ethacrylate, tert-butyl(meth)acrylate, tert-butyl ethacrylate, n-octyl(meth)acrylate, ethylhexyl(meth)acrylate, stearyl(meth)acrylate, lauryl(meth)acrylate, etc. and mixtures thereof.

Suitable monomers S.3) are also acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide, N-(n-octyl)(meth)acrylamide, N-ethylhexyl(meth)acrylamide, N-stearyl(meth)acrylamide, N-lauryl(meth)acrylamide, etc. and mixtures thereof.

Furthermore, the polymers preferably comprise at least one monomer S.4) in copolymerized form which is chosen from compounds of the general formula IV

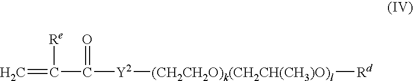

in which
the order of the alkylene oxide units is arbitrary,
k and l, independently of one another, are an integer from 0 to 1000, where the sum of k and l is at least 5,
R^{d} is hydrogen, C₁-C₃₀-alkyl or C₅-C₈-cycloalkyl,
R^{e} is hydrogen or C₁-C₈-alkyl,
Y² is O or NR^{f}, where R^{f} is hydrogen, C₁-C₃₀-alkyl or C₅-C₈-cycloalkyl.

Preferably, k in the formula IV is an integer from 1 to 500, in particular 3 to 250. Preferably, l is an integer from 0 to 100.

Preferably, R^{e} is hydrogen, methyl, ethyl, n-propyl, isopropyl, n-butyl, sec-butyl, tert-butyl, n-pentyl or n-hexyl, in particular hydrogen, methyl or ethyl.

Preferably, R^{d} in the formula IV is hydrogen, methyl, ethyl, n-propyl, isopropyl, n-butyl, sec-butyl, n-pentyl, n-hexyl, octyl, 2-ethylhexyl, decyl, lauryl, palmityl or stearyl.

Preferably, Y² in the formula IV is O or NH.

Preferably, the acid-group-containing polymer S) comprises
acrylic acid and/or methacrylic acid,
at least one C₁-C₄-alkyl(meth)acrylate which is preferably chosen from methyl methacrylate, ethyl acrylate, ethyl methacrylate, tert-butyl acrylate and mixtures thereof,
at least one C₁-C₄-alkyl(meth)acrylamide, and
if appropriate at least one crosslinker in copolymerized form.

Furthermore, the acid-group-containing polymer S) preferably comprises
acrylic acid and/or methacrylic acid,
at least one monomer which is chosen from C₁-C₄-alkyl(meth)acrylates, C₁-C₄-alkyl(meth)acrylamides and mixtures thereof,
at least one polyether acrylate, and
if appropriate at least one crosslinker in copolymerized form.

Furthermore, the acid-group-containing polymer S) preferably comprises
t-butyl acrylate and/or ethyl acrylate, and
acrylic acid and/or methacrylic acid
   in copolymerized form.

Furthermore, the acid-group-containing polymer S) preferably comprises
at least one C₁-C₄-alkyl(meth)acrylate, preferably t-butyl acrylate,
vinylpyrrolidone and/or vinylcaprolactam, and
acrylic acid and/or methacrylic acid
   in copolymerized form.

Anionic polymers preferred as polymers S) are, for example, homopolymers and copolymers of acrylic acid and methacrylic acid and salts thereof. These also include crosslinked polymers of acrylic acid, as are obtainable under the INCI name Carbomer. Such crosslinked homopolymers of acrylic acid are commercially available, for example, under the name Carbopol® from BF GOODRICH. Preference is also given to hydrophobically modified crosslinked polyacrylate polymers, such as Carbopol® Ultrez 21 from Noveon.

Compositions based on homopolymers and copolymers of acrylic acid and methacrylic acid are advantageously suitable for formulation as gels, for example for setting gels, and also for formulation of foams.

Further examples of suitable anionic polymers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm und Haas. Particularly suitable polymers are also copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and, if appropriate, further vinyl esters (e.g. Luviset® grades), maleic anhydride copolymers, if appropriate reacted with alcohol, anionic polysiloxanes, e.g. carboxyfunctional polysiloxanes, t-butyl acrylate, methacrylic acid (e.g. Luviskol® VBM), copolymers of acrylic acid and methacrylic acid with hydrophobic monomers, such as, for example, C₄-C₃₀-alkyl esters of (meth)acrylic acid, C₄-C₃₀-alkylvinyl esters, C₄-C₃₀-alkyl vinyl ethers and hyaluronic acid. Examples of anionic polymers are also vinyl acetate/crotonic acid copolymers, as are commercially available, for example, under the names Resyn® (National Starch) and Gafset® (GAF), and vinylpyrrolidone/vinyl acrylate copolymers, obtainable for example under the trade name Luviflex® (BASF). Further suitable polymers are the vinylpyrrolidone/acrylate terpolymer available under the name Luviflex® VBM-35 (BASF) and polyamides containing sodium sulfonate, or polyesters containing sodium sulfonate.

The group of suitable anionic polymers also comprises, for example, Balance® CR (National Starch; acrylate copolymer), Balance® 0/55 (National Starch; acrylate copolymer), Balance® 47 (National Starch; octylacrylamide/acrylate/butylaminoethyl methacrylate copolymer), Aquaflex® FX 64 (ISP; isobutylene/ethylmaleimide/hydroxy-ethylmaleimide copolymer), Aquaflex® SF-40 (ISP/National Starch; VP/vinylcaprolactam/DMAPA acrylate copolymer), Allianz® LT-120 (ISP/Rohm & Haas; acrylate/C1-2 succinate/hydroxyacrylate copolymer), Aquarez® HS (Eastman; Polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethyl ester of poly(methyl vinyl ether/maleic acid in ethanol), Amphomer® HC (National Starch; acrylate/octylacrylamide copolymer), Amphomer® 28-4910 (National Starch; octylacrylamide/acrylate/butylaminoethyl methacrylate copolymer), Advantage® HC 37 (ISP; terpolymer of vinylcaprolactam/vinylpyrrolidone/dimethylaminoethyl ethacrylate), Advantage® LC55 and LC80 or LC A and LC E, Advantage® Plus (ISP; VA/butyl maleate/isobornyl acrylate copolymer), Acudyne® 258 (Rohm & Haas; acrylate/hydroxy ester acrylate copolymer), Luviset® P.U.R. (BASF, polyurethane-1), Luviflex® Silk (BASF), Eastman® AQ 48 (Eastman), Styleze®CC-10 (ISP; VP/DMAPA acrylates copolymer), Styleze® 2000 (ISP; VP/acrylates/lauryl methacrylate copolymer), DynamX (National Starch; polyurethane-14 AMP-acrylates copolymer), Resyn XP (National Starch; acrylates/octylacrylamide copolymer), Fixomer A-30 (Ondeo Nalco; polymethacrylic acid (and) acrylamidomethylpropanesulfonic acid), Fixate G-100 (Noveon; AMP-acrylates/allyl methacrylate copolymer).

Suitable copolymers S) are also the terpolymers, described in U.S. Pat. No. 3,405,084, of vinylpyrrolidone, C₁-C₁₀-alkyl, cycloalkyl and aryl(meth)acrylates and acrylic acid. Suitable copolymers S) are also the terpolymers, described in EP-A-0 257 444 and EP-A-0 480 280, of vinylpyrrolidone, tert-butyl(meth)acrylate and (meth)acrylic acid. Suitable copolymers S) are also the copolymers described in DE-A-42 23 066 which comprise at least one (meth)acrylic ester, (meth)acrylic acid and N-vinylpyrrolidone and/or N-vinylcaprolactam in copolymerized form. Reference is made here to the disclosure of these documents.

The abovementioned polymers S) are prepared by known methods, for example solution polymerization, precipitation polymerization, suspension polymerization or emulsion polymerization.

The polyurethanes used according to the invention preferably have K values (measured in accordance with E. Fikentscher, Cellulose-Chemie 13 (1932), pp. 58-64, on a 1% strength solution in N-methylpyrrolidone) in a range from 15 to 90, preferably 20 to 60.

To prepare the polymer component A), a corresponding polymer component which has free acid groups (anionogenic groups) is subjected to a neutralization with at least one inorganic base and at least one organic base. The neutralization of the acid groups can take place successively with, in each case, a single base or mixtures of more than one base, or with the help of a mixture of all of these bases to be used for the neutralization. Neutralization preferably takes place successively according to base strength, with the strongest base firstly being used, followed by the weaker in each case. For the neutralization, the inorganic bases are preferably used in the form of a solution in an aqueous medium. Suitable aqueous media are water and mixtures of water and at least one water-miscible solvent, e.g. an alkanol, such as methanol, ethanol, propanol, isopropanol, etc. Organic bases liquid under normal conditions can be used without diluent for the neutralization. In general, the organic bases are also used in an aqueous medium for the neutralization.

Cosmetic and pharmaceutical compositions based on the polymer component according to the invention have, as component B), at least one cosmetically or pharmaceutically acceptable active ingredient or auxiliary.

The components B) are chosen according to the desired field of use of the composition. Besides components typical of the field of use (e.g. certain pharmaceutical active ingredients), they are chosen from carriers, excipients, emulsifiers, surfactants, preservatives, fragrances, polymers different from component A), gel formers, dyes, pigments, photoprotective agents, consistency regulators, antioxidants, antifoams, antistats, resins, solvents, solubility promoters, stabilizers, sterilizing agents, propellants, drying agents, opacifiers, etc.

Preferably, the compositions have a carrier component B) which is chosen from water, hydrophilic components, hydrophobic components and mixtures thereof.

Suitable hydrophilic carriers B) are, for example, water, mono-, di- or polyhydric alcohols having preferably 1 to 8 carbon atoms, such as ethanol, n-propanol, isopropanol, propylene glycol, glycerol, sorbitol, etc. Preferably, the hydrophilic carrier used is water or a mixture of water with at least one water-miscible organic solvent, preferably at least one C₂-C₄-alkanol, in particular ethanol.

Suitable hydrophobic carriers B) are, for example, chosen from
i) oils, fats, waxes,
ii) esters of C₆-C₃₀-monocarboxylic acids with mono, di- or trihydric alcohols different from i),
iii) saturated acyclic and cyclic hydrocarbons,
iv) fatty acids,
v) fatty alcohols,
vi) propellant gases,
   and mixtures thereof.

The compositions according to the invention have, for example, an oil or fat component B) which is chosen from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil or fat components B) are chosen from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, pig grease, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candelilla wax, spermaceti, and mixtures of the abovementioned oil and/or fat components.

Suitable cosmetically and pharmaceutically compatible oil and fat components B) are described in Karl-Heinz Schrader, Grundlagen und Rezepturen der Kosmetika [Fundamentals and formulations of cosmetics], 2nd Edition, Verlag Hüthig, Heidelberg, pp. 319-355, to which reference is hereby made.

The cosmetic compositions according to the invention may be hair cosmetic, skin cosmetic, dermatological, hygiene or pharmaceutical compositions. On account of their film-forming and flexible properties, the crosslinked polyurethanes described above are particularly preferably suitable as additives for hair and skin cosmetics, in particular for hair cosmetics and specifically as hair-setting compositions.

Preferably, the compositions according to the invention are in the form of a spray, gel, foam, ointment, cream, emulsion, suspension, lotion, milk or paste. If desired, liposomes or microspheres can also be used.

The cosmetically or pharmaceutically active compositions according to the invention can additionally comprise cosmetically and/or dermatologically active ingredients, and auxiliaries B).

Preferably, the cosmetic compositions according to the invention comprise at least one crosslinked polyurethane as defined above, at least one carrier as defined above and at least one constituent different therefrom which is preferably chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolyzates, lipids, antioxidants, antifoams, antistats, emollients and softeners.

Customary thickeners in such formulations are crosslinked polyacrylic acids and derivatives thereof (if not already present as component A)), polysaccharides and derivatives thereof, such as xanthan gum, agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone.

Suitable cosmetically and/or dermatologically active ingredients are, for example, coloring active ingredients, skin and hair pigmentation agents, tinting agents, tanning agents, bleaches, keratin-hardening substances, antimicrobial active ingredients, photofilter active ingredients, repellent active ingredients, hyperemic substances, keratolytic and keratoplastic substances, antidandruff active ingredients, antiphlogistics, keratinizing substances, active ingredients which have an antioxidative effect or act as free-radical scavengers, skin-moisturizing or humectant substances, refatting active ingredients, deodorizing active ingredients, sebostatic active ingredients, plant extracts, antierythematous or antiallergic active ingredients, and mixtures thereof.

Artificially skin-tanning active ingredients which are suitable for tanning the skin without natural or artificial irradiation with UV rays are, for example, dihydroxyacetone, alloxan and walnut shell extract. Suitable keratin-hardening substances are usually active ingredients as are also used in antiperspirants, such as, for example, potassium aluminum sulfate, aluminum hydroxychloride, aluminum lactate, etc. Antimicrobial active ingredients are used to destroy microorganisms or to inhibit their growth and thus serve both as preservatives and as deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable photofilter active ingredients are substances which absorb UV rays in the UV-B and/or UV-A region. Suitable UV filters are, for example, 2,4,6-triaryl-1,3,5-triazines, in which the aryl groups can each carry at least one substituent which is preferably chosen from hydroxy, alkoxy, specifically methoxy, alkoxycarbonyl, specifically methoxycarbonyl and ethoxycarbonyl and mixtures thereof. Also suitable are p-aminobenzoic esters, cinnamic esters, benzophenones, camphor derivatives, and pigments which stop UV rays, such as titanium dioxide, talc and zinc oxide. Suitable repellent active ingredients are compounds which are able to repel or drive away certain animals, in particular insects, from people. These include, for example, 2-ethyl-1,3-hexanediol, N,N-diethyl-m-toluamide etc. Suitable hyperemic substances, which stimulate blood flow through the skin, are, for example, essential oils, such as pine, lavender, rosemary, juniperberry, horsechestnut extract, birch leaf extract, hayflower extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counteract skin irritations are, for example, allantoin, bisabolol, dragosantol, camomile extract, panthenol, etc.

The cosmetic compositions according to the invention can comprise, as cosmetic and/or pharmaceutical active ingredient (and also, if appropriate, as auxiliary), at least one cosmetically or pharmaceutically acceptable polymer which differs from the polymers present in polymer component A). These include, quite generally, anionic, cationic, amphoteric and neutral polymers.

Examples of anionic polymers are the polymers specified above for component A) provided they are used in the salt form.

Suitable cationic polymers are, for example, cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinyl-imidazolium salts (Luviquat® Hold); cationic cellulose derivatives (Polyquaternium-4 and -10), acrylamido copolymers (Polyquaternium-7) and chitosan. Suitable cationic (quaternized) polymers are also Merquat® (polymer based on dimethyldiallylammonium chloride), Gafquat® (quaternary polymers which are formed by the reaction of polyvinylpyrrolidone with quaternary ammonium compounds), polymer JR (hydroxyethylcellulose with cationic groups) and plant-based cationic polymers, e.g. guar polymers, such as the Jaguar® grades from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex® Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers available under the names Amphomer® (National Starch), and zwitterionic polymers, as disclosed, for example, in the German patent applications DE 39 29 973, DE 21 50 557, DE 28 17 369 and DE 37 08 451. Acrylamidopropyl-trimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers, which are commercially available under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or -dispersible polymers, e.g. polyether siloxanes, such as Tegopren® (Goldschmidt) or Belsil® (Wacker).

The formulation base of pharmaceutical compositions according to the invention preferably comprises pharmaceutically acceptable auxiliaries. Of pharmaceutical acceptability are the auxiliaries which are known for use in the field of pharmacy, food technology and related fields, in particular those listed in relevant pharmacopoeia (e.g. DAB Ph. Eur. BP NF), and other auxiliaries whose properties do not preclude a physiological application.

Suitable auxiliaries may be: glidants, wetting agents, emulsifying and suspending agents, preservatives, antioxidants, antiirritatives, chelating agents, emulsion stabilizers, film formers, gel formers, odor masking agents, resins, hydrocolloids, solvents, solubility promoters, neutralizing agents, permeation accelerators, pigments, quaternary ammonium compounds, refatting and superfatting agents, ointment, cream or oil bases, silicone derivatives, stabilizers, sterilizing agents, propellants, drying agents, opacifiers, thickeners, waxes, softeners, white oils. An embodiment with regard to this is based on expert knowledge, as is presented, for example, in Fiedler, H. P. Lexikon der Hilfsstoffe für Pharmazie, Kosmetik und angrenzende Gebiete [Lexicon of auxiliaries for pharmacy, cosmetics and related fields], 4th edition, Aulendorf: ECV-Editio-Kantor-Verlag, 1996.

To produce the dermatological compositions according to the invention, the active ingredients can be mixed or diluted with a suitable auxiliary (excipient). Excipients may be solid, semisolid or liquid materials which can serve as vehicles, carriers or medium for the active ingredient. The admixing of further ingredients takes place, if desired, in the manner known to the person skilled in the art. In addition, the polymer component used according to the invention is suitable as auxiliary in pharmacy, preferably as or in (a) coating composition(s) or binder(s) for solid drug forms. It can also be used in creams and as tablet coatings and tablet binders.

According to a preferred embodiment, the compositions according to the invention are a skin-cleansing composition.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and gels, foam baths, oil baths and scrub preparations, shaving foams, lotions and creams.

According to a further preferred embodiment, the compositions according to the invention are cosmetic compositions for the care and protection of the skin, nailcare compositions or preparations for decorative cosmetics.

Suitable skin cosmetic compositions are, for example, face tonics, face masks, deodorants and other cosmetic lotions. Compositions for use in decorative cosmetics comprise, for example, concealing sticks, stage make-up, mascara and eyeshadows, lipsticks, kohl pencils, eyeliners, blushers, powder and eyebrow pencils.

Furthermore, the polymer component according to the invention can be used in nose strips for pore cleansing, in antiacne compositions, repellents, shaving compositions, hair-removal compositions, intimate care compositions, footcare compositions, and in babycare.

The skincare compositions according to the invention are, in particular, W/O or O/W skin creams, day and night creams, eye creams, face creams, antiwrinkle creams, moisturizing creams, bleaching creams, vitamin creams, skin lotions, care lotions and moisturizing lotions.

Skin cosmetic and dermatological compositions based on the polymer component according to the invention described above exhibit advantageous effects. The polymers can, inter alia, contribute to the moisturization and conditioning of the skin and to the improvement in the feel of the skin. The polymers can also act as thickeners in the formulations. By adding the polymers according to the invention, a considerable improvement in skin compatibility can be achieved in certain formulations.

Skin cosmetic and dermatological compositions preferably comprise at least one polymer component according to the invention in an amount of from about 0.001 to 30% by weight, preferably 0.01 to 20% by weight, very particularly preferably 0.1 to 12% by weight, based on the total weight of the composition.

Particularly photoprotective compositions based on the polymer component according to the invention have the property of increasing the residence time of the UV-absorbing ingredients compared to customary auxiliaries such as polyvinylpyrrolidone.

Depending on the field of use, the compositions according to the invention can be applied in a form suitable for skincare, such as, for example, as cream, foam, gel, stick, mousse, milk, spray (pump spray or propellant-containing spray) or lotion.

Besides the polymer component according to the invention and suitable carriers, the skin cosmetic preparations can also comprise further active ingredients and auxiliaries customary in skin cosmetics, as described above. These preferably include emulsifiers, preservatives, perfume oils, cosmetic active ingredients, such as phytantriol, vitamin A, E and C, retinol, bisabolol, panthenol, photoprotective agents, bleaches, colorants, tinting agents, tanning agents, collagen, protein hydrolyzates, stabilizers, pH regulators, dyes, salts, thickeners, gel formers, consistency regulators, silicones, humectants, refatting agents and further customary additives.

Preferred oil and fat components of the skin cosmetic and dermatological compositions are the abovementioned mineral and synthetic oils, such as, for example, paraffins, silicone oils and aliphatic hydrocarbons having more than 8 carbon atoms, animal and vegetable oils; such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin, and mixtures thereof.

The polymer component according to the invention can also be mixed with conventional polymers if specific properties are to be established.

To establish certain properties, such as, for example, improvement in the feel to the touch, the spreading behavior, the water resistance and/or the binding of active ingredients and auxiliaries, such as pigments, the skin cosmetic and dermatological preparations can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

The cosmetic or dermatological preparations are prepared in accordance with customary methods known to the person skilled in the art.

Preferably, the cosmetic and dermatological compositions are in the form of emulsions, in particular in the form of water-in-oil (W/O) or oil-in-water (O/W) emulsions. However, it is also possible to choose other types of formulation, for example hydrodispersions, gels, oils, oleogels, multiple emulsions, for example in the form of W/O/W or O/W/O emulsions, anhydrous ointments or ointment bases, etc.

The preparation of emulsions takes place by known methods. Besides at least one polymer component according to the invention, the emulsions generally comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The choice of additives specific to the type of emulsion and the preparation of suitable emulsions is described, for example, in Schrader, Grundlagen und Rezepturen der Kosmetika [Fundamentals and formulations of cosmetics], Hüthig Buch Verlag, Heidelberg, 2nd edition, 1989, third part, to which reference is hereby expressly made.

A suitable emulsion, e.g. for a skin cream etc., generally comprises an aqueous phase which is emulsified in an oil or fat phase by means of a suitable emulsifier system. To provide the aqueous phase, a polymer component according to the invention can be used.

Preferred fatty components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophyllum oil, lanolin and derivatives thereof, ricinus oil, sesame oil, olive oil, jojoba oil, karite oil, hoplostethus oil; mineral oils whose distillation start-point under atmospheric pressure is at about 250° C. and whose distillation end-point is at 410° C., such as, for example, vaseline oil; esters of saturated or unsaturated fatty acids, such as alkyl myristates, e.g. isopropyl, butyl or cetyl myristate, hexadecyl stearate, ethyl or isopropyl palmitate, octanoic or decanoic acid triglycerides and cetyl ricinoleate.

The fatty phase can also comprise silicone oils which are soluble in other oils, such as dimethylpolysiloxane, methylphenylpolysiloxane and the silicone glycol copolymer, fatty acids and fatty, alcohols.

Besides the polymer component according to the invention, it is also possible to use waxes, such as, for example, carnauba wax, candelilla wax, beeswax, microcrystalline wax, ozokerite wax, and Ca, Mg and Al oleates, myristates, linoleates and stearates.

In addition, a composition according to the invention can be in the form of the O/W emulsion. Such a type of emulsion usually comprises an oil phase, emulsifiers which stabilize the oil phase in the water phase, and an aqueous phase, which is usually in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one polymer component according to the invention, and usually anionic surfactants as base surfactants and amphoteric and/or non-ionic surfactants as cosurfactants. Further suitable active ingredients and/or auxiliaries are generally chosen from lipids, perfume oils, dyes, organic acids, preservatives and antioxidants, and thickeners/gel formers, skin conditioners and humectants.

These formulations preferably comprise 2 to 50% by weight, preferably 5 to 40% by weight, particularly preferably 8 to 30% by weight, of surfactants, based on the total weight of the formulation.

All of the anionic, neutral, amphoteric or cationic surfactants customarily used in body-cleansing compositions may be used in the washing, showering and bath preparations.

Suitable anionic surfactants are, for example, alkyl sulfates, alkyl ether sulfates, alkyl-sulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mole of alcohol. Also suitable are alkylamine oxides, mono- or dialkylalkanolamides, fatty acid esters of polyethylene glycols, ethoxylated fatty acid amides, alkyl polyglycosides or sorbitan ether esters.

Furthermore, the washing, showering and bath preparations can comprise customary cationic surfactants, such as, for example, quaternary ammonium compounds, for example cetyltrimethylammonium chloride.

Furthermore, the shower gel/shampoo formulations can comprise thickeners, such as, for example, sodium chloride, PEG-55, propylene glycol oleate, PEG-120 methylglucose dioleate and others, and also preservatives, further active ingredients and auxiliaries and water.

According to a particularly preferred embodiment, the compositions according to the invention are a hair-treatment composition.

Hair-treatment compositions according to the invention preferably comprise at least one polymer component according to the invention in an amount in the range from about 0.1 to 30% by weight, preferably 0.5 to 20% by weight, based on the total weight of the composition.

Preferably, the hair-treatment compositions according to the invention are in the form of a hairspray, setting foam, hair mousse, hair gel, shampoo, hair foam, end fluid, neutralizer for permanent waves, hair colorant and bleach or hot-oil treatment. Depending on the field of use, the hair cosmetic preparations can be applied in the form of an (aerosol) spray, (aerosol) foam, gel, gel spray, cream, lotion or wax. Hairsprays comprise here both aerosol sprays and also pump sprays without propellant gas. Hair foams comprise both aerosol foams and also pump foams without propellant gas. Hairsprays and hair foams preferably comprise predominantly or exclusively water-soluble or water-dispersible components. If the compounds used in the hairsprays and hair foams according to the invention are water-dispersible, they can be applied in the form of aqueous microdispersions with particle diameters of usually 1 to 350 nm, preferably 1 to 250 nm. The solids contents of these preparations here are usually in a range from about 0.5 to 20% by weight. These microdispersions generally require no emulsifiers or surfactants for their stabilization.

The hair cosmetic formulations according to the invention comprise, in a preferred embodiment,
0.2 to 10% by weight of at least one polymer component according to the invention,
40 to 99% by weight of water and/or alcohol,
0 to 45% by weight of at least one propellant gas,
0 to 20% by weight of at least one surface-active compound,
0 to 3% by weight of at least one UV absorber, and
up to 5% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolyzates, alpha- and beta-hydroxycarboxylic acids, stabilizers, pH regulators, dyes, viscosity regulators, gel formers, salts, humectants, refatting agents, complexing agents and further customary additives.

Also included here are all styling and conditioner polymers known in cosmetics, which can be used in combination with the polymers according to the invention if very specific properties are to be established.

Suitable conventional hair cosmetic polymers are, for example, the abovementioned cationic, anionic, neutral, nonionic and amphoteric polymers, to which reference is made here.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (ETFA) and amino-functional silicone compounds such as amodimethicones (CTFA).

The polymers according to the invention are particularly suitable as setting agents in hairstyling preparations, in particular hairsprays (aerosol sprays and pump sprays without propellant gas) and hair foams (aerosol foams and pump foams without propellant gas).

In one preferred embodiment, spray preparations comprise
a) 0.1 to 10% by weight of at least one polymer component according to the invention,
b) 20 to 99.9% by weight of water and/or alcohol,
c) 0 to 70% by weight of at least one propellant,
d) 0 to 20% by weight of further constituents.

Propellants are the propellants customarily used for hairsprays or aerosol foams. Preference is given to mixtures of propane/butane, pentane, dimethyl ether, 1,1-difluoroethane (HFC-152 a), carbon dioxide, nitrogen or compressed air.

A formulation preferred according to the invention for aerosol hair foams comprises
a) 0.1 to 10% by weight of at least one polymer component according to the invention,
b) 55 to 99.8% by weight of water and/or alcohol,
c) 5 to 20% by weight of a propellant,
d) 0.1 to 5% by weight of an emulsifier,
e) 0 to 10% by weight of further constituents.

Emulsifiers which can be used are all emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
a) 0.1 to 10% by weight of at least one polymer component according to the invention,
b) 80 to 99.85% by weight of water,
c) 0 to 30% by weight of at least one alcohol, in particular ethanol,
d) 0 to 3% by weight, preferably 0.05 to 2% by weight, of a gel former,
e) 0 to 20% by weight of further constituents.

During the preparation of gels based on the polymer component according to the invention, it is possible to use customary gel formers, for example, in order to establish specific rheological or other application-related properties of the gels. Gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, poly-saccharides, e.g. xanthan gum, caprylic/capric triglyceride, sodium acrylate copolymers, Polyquaternium-32 (and) Paraffinum Liquidum (INCI), sodium acrylate copolymers (and) paraffinum liquidum (and) PPG-1 trideceth-6, acrylamidopropyltrimonium chloride/acrylamide copolymers, steareth-10 allyl ether acrylate copolymers, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium-44. Crosslinked homopolymers of acrylic acid suitable as gel formers are, for example, commercially available under the name Carbopol® from BF GOODRICH. Preference is also given to hydrophobically modified crosslinked poly-acrylate polymers, such as Carbopol® Ultrez 21 from Noveon. Further examples of anionic polymers suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm und Haas.

The polymer component according to the invention can be used in cosmetic preparations as conditioner.

The polymer component according to the invention can preferably be used in shampoo formulations as setting agent and/or conditioner. Preferred shampoo formulations comprise
a) 0.05 to 10% by weight of at least one polymer component according to the invention,
b) 25 to 94.95% by weight of water,
c) 5 to 50% by weight of surfactants,
d) 0 to 5% by weight of a further conditioner,
e) 0 to 10% by weight of further cosmetic constituents.

All anionic, neutral, amphoteric or cationic surfactants used customarily in shampoos can be used in the shampoo formulations.

Suitable anionic surfactants are, for example, alkyl sulfates, alkyl ether sulfates, alkyl-sulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate, for example, are suitable.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethyl-ammonium chloride.

In the shampoo formulations, to achieve certain effects, customary conditioners can be used in combination with the crosslinked polyurethanes. These include, for example, the abovementioned cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylamino-ethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Poly-quaternium-7). In addition, protein hydrolyzates can be used, as can conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino-functional silicone compounds such as amodimethicones (CTFA). In addition, cationic guar derivatives, such as Guar hydroxypropyltrimonium chloride (INCI) can be used.

The invention further provides the use of a polymer component according to the invention, as defined above, as auxiliary in pharmacy, preferably as or in (a) coating(s) for solid drug forms, for modifying rheological properties, as surface-active compound, as or in (an) adhesive(s), and as or in (a) coating(s) for the textile, paper, printing and leather industries.

The invention is explained in more detail by reference to the following nonlimiting examples.

### EXAMPLES

### I. Preparation of Polyurethanes

### Linear Polyurethane PU 1

In a 4-necked flask which was equipped with stirrer, dropping funnel, thermometer, reflux condenser and a device for working under nitrogen, 1 mol of a polyesterdiol (Mₙ=1000 g/mol), 2.5 mol of dimethylolpropanoic acid, 1.2 mol of neopentyl glycol and 0.85 g of DABCO (1,4-diazabicyclo[2.2.2]octane) in 690 g of methyl ethyl ketone were heated to a temperature of about 60° C. and dissolved with stirring. Then, at this temperature and with stirring, 5 mol of isophorone diisocyanate was metered in such that the reaction temperature remained below 90° C. The reaction mixture was then after-stirred at about 80° C. for about a further three hours until the NCO content remained virtually constant, and the mixture is then left to cool to a temperature of 40° C. The polyurethane obtained was neutralized with a neutralizing agent according to table 2. The solvent was then distilled off under reduced pressure at 40° C. Pulverulent products can be obtained by spray-drying.

The linear polyurethane PU 2 was prepared analogously (see table 1).

Crosslinked Polyurethane PU 3

In a 4-necked flask which was equipped with stirrer, dropping funnel, thermometer, reflux condenser and a device for working under nitrogen, 0.5 mol of polytetrahydrofuran (Mₙ=1000 g/mol), 0.3 mol of trimethylolpropane, 2.7 mol of dimethylolpropanoic acid, 1.5 mol of neopentyl glycol and 0.85 g of DABCO were dissolved in 690 g of methyl ethyl ketone with heating to a temperature of about 60° C. and with stirring. As soon as all of the components had dissolved, a mixture of 1 mol of hexamethylene diisocyanate and 4 mol of isophorone diisocyanate was metered in, at this temperature and with stirring, such that the reaction temperature remained below 90° C. The reaction mixture was then after-stirred at about 80° C. for about a further three hours until the NCO content remained virtually constant, and the mixture was then left to cool to a temperature of 40° C. The polyurethane obtained was neutralized with a neutralizing agent according to table 2. The solvent was then distilled off under reduced pressure at 40° C. Pulverulent products can be obtained by spray-drying.

The crosslinked polyurethane PU 4 was prepared analogously (see table 1).

TABLE 1

| | PE-Diol | | | | | | | |
|---|---|---|---|---|---|---|---|---|
| Polymer | 1000 | PTHF 1000 | NPG | DMPA | TMP | IPDI | HDI | K value*) |
| Linear | 1 | — | 1.2 | 2.5 | — | 5 | — | 30 |
| PU 1 | | | | | | | | |
| Linear | — | 1 | 1.3 | 2.5 | — | 5 | — | 30 |
| PU 2 | | | | | | | | |
| crosslinked | — | 0.5 | 1.5 | 2.7 | 0.3 | 4 | 1 | 40 |
| PU 3 | | | | | | | | |
| crosslinked | — | 0.5 | 1.7 | 2.5 | 0.4 | 4.2 | 0.8 | 38.5 |
| PU 4 | | | | | | | | |

PE-Diol 1000 = polyesterdiol of isophthalic acid, adipic acid and hexanediol, Mₙ = 1000 g/mol
PTHF 1000 = polytetrahydrofuran, MG = 1000 g/mol
NPG = neopentyl glycol
DMPA = dimethylolpropanoic acid
TMP = trimethylolpropane
IPDI = isophorone diisocyanate
HDI = hexamethylene diisocyanate
*)The K value was determined on a 1% strength solution in N-methyl-2-pyrrolidinone.

### II. Application Properties

The polyurethanes described in table 1 were neutralized with the neutralizing agents given in table 2 and have the following mechanical properties which are summarized in table 2.

The test was based on the following evaluation scale:

| Grade | Clarity | Stickiness | Elasticity |
|---|---|---|---|
| 1 | clear | not sticky | very flexible |
| 2 | slightly cloudy | slightly sticky | flexible |
| 3 | cloudy | sticky | extensible |
| 4 | — | — | not very flexible |
| 5 | — | — | brittle |

TABLE 2

| | Neutralizing | | | | | |
|---|---|---|---|---|---|---|
| | agent/degree | Polymer | | | | |
| Example | of neutralization | (as in table 1) | Hardness | Clarity | Stickiness | Elasticity |
| 1 | AMP | linear PU 1 | very brittle | 1 | 1 | 5 |
| (comparison) | 100% | | | | | |
| 2 | KOH | linear PU 1 | hard, | 1 | 1 | 4-5 |
| (comparison) | 100% | | brittle | | | |
| 3 | TEA | linear PU 1 | soft, | 1 | 2 | 1 |
| (comparison) | 100% | | flexible | | | |
| 4 | KOH:TEA | linear PU 1 | moderately | 1 | 2 | 3 |
| | 50%:50% | | soft | | | |
| 5 | KOH | linear PU 2 | moderately | 1 | 1 | 4 |
| (comparison) | 100% | | hard | | | |
| 6 | KOH:TEA | linear PU 2 | soft, | 1 | 2 | 1 |
| | 50%:50% | | flexible | | | |
| 7 | KOH | crosslinked PU 3 | hard, solid | 1 | 1 | 3-4 |
| (comparison) | 100% | | | | | |
| 8 | KOH:TEA | crosslinked PU 3 | very soft | — | 2 | 1 |
| | 50%:50% | | | | | |
| 9 | KOH:AMP | crosslinked PU 3 | hard, solid | 1 | 1 | 2 |
| | 50%:50% | | | | | |
| 10 | KOH | crosslinked PU 4 | hard, solid | — | 1 | 5 |
| (comparison) | 100% | | | | | |
| 11 | TEA | crosslinked PU 4 | very soft | — | 3 | 1 |
| (comparison) | 100% | | | | | |
| 12 | | Mixture of Ex. 10 | hard, solid | — | 1 | 4 |
| | | and Ex. 11 | | | | |
| | | [95:5 w/w] | | | | |
| 13 | | Mixture of | hard, solid | — | 1 | 3-4 |
| | | Ex. 10 and Ex. 11 | | | | |
| | | [90:10 w/w] | | | | |
| 14 | | Mixture of | hard, solid | — | 2 | 3-4 |
| | | Ex. 10 and Ex. 11 | | | | |
| | | [85:15 w/w] | | | | |
| 15 | | Mixture of | soft | 1 | 2 | 2 |
| | | Ex. 10 and Ex. 11 | flexible | | | |
| | | [50:50 w/w] | | | | |

AMP = amino-2-methylpropanol
KOH = potassium hydroxide
TEA = triethanolamine

### III. Application Examples

### Example 1

| VOC 80 aerosol hairspray | [%] |
|---|---|
| Polymer component from example No. 4 (25% strength solution) | 12.00 |
| Water | 8.00 |
| Dimethyl ether | 40.00 |
| Ethanol | 40.00 |
| Further additive: silicone, perfume, antifoam . . . | |

The example can be repeated with the polymer components from example No. 6, 8, 9, 12, 13, 14 and 15. In each case, a VOC 80 aerosol hairspray with good properties is obtained.

### Example 2

| VOC 55 aerosol hairspray | [%] |
|---|---|
| Polymer component from example No. 4 (25% strength solution) | 12.00 |
| Water | 33.00 |
| Dimethyl ether | 40.00 |
| Ethanol | 15.00 |
| Further additive: silicone, perfume, antifoam . . . | |

The example can be repeated with the polymer components from example No. 6, 8, 9, 12, 13, 14 and 15. In each case, a VOC 55 aerosol hairspray with good properties is obtained.

### Example 3

| VOC 55 aerosol hairspray | [%] |
|---|---|
| Polymer component from example No. 4 | 10.00 |
| (25% strength solution) | |
| Ultrahold Strong (BASF) | 1.00 |
| Water | 34.00 |
| Dimethyl ether | 40.00 |
| Ethanol | 15.00 |
| +AMP | to pH 8.3 |
| Further additive: silicone, perfume, antifoam . . . | |

The example can be repeated with the polymer components from example No. 6, 8, 9, 12, 13, 14 and 15. In each case, a VOC 55 aerosol hairspray with good properties is obtained.

### Example 4

| VOC 55 aerosol hairspray | [%] |
|---|---|
| Polymer component from example No. 4 | 8.00 |
| (25% strength solution) | |
| Stepanhold R-1 *⁾ (Stepan Chemical Co.) | 1.00 |
| Water | 36.00 |
| Dimethyl ether | 40.00 |
| Ethanol | 15.00 |
| +AMP | to pH 8.3 |
| Further additive: silicone, perfume, antifoam . . . | |

*⁾Stepanhold R-1 = poly(vinylpyrrolidone/ethyl methacrylate/methacrylic acid)

The example can be repeated with the polymer components from example No. 6, 8, 9, 12, 13, 14 and 15. In each case, a VOC 55 aerosol hairspray with good properties is obtained.

### Example 5

| VOC 55 hand pump spray | [%] |
|---|---|
| Polymer component from example No. 4 (25% strength solution) | 12.00 |
| Water | 33.00 |
| Ethanol | 55.00 |
| Further additive: silicone, perfume, antifoam . . . | |

The example can be repeated with the polymer components from example No. 6, 8, 9, 12, 13, 14 and 15. In each case, a VOC 55 hand pump spray with good properties is obtained.

### Example 6

| Aqueous hand pump spray | [%] |
|---|---|
| Polymer component from example No. 4 (25% strength solution) | 10.00 |
| Luviset Clear *⁾ (20% strength solution) | 5.00 |
| Water | 85.00 |
| Further additive: water-soluble silicone, perfume, antifoam . . . | |

*⁾ Luviset Clear: poly(vinylpyrrolidone/methacrylamide/vinylimidazole), BASF

The example can be repeated with the polymer components from example No. 6, 8, 9, 12, 13, 14 and 15. In each case, an aqueous hand pump spray with good properties is obtained.

### Example 7

| Foam conditioner | [%] |
|---|---|
| Polymer component from example No. 4 | 20.00 |
| (25% strength aqueous solution) | |
| Cremophor A 25 (Ceteareth 25/BASF) | 0.2 |
| Comperlan KD (Coamide DEA/Henkel) | 0.1 |
| Water | 69.7 |
| Propane/butane | 10.0 |
| Further additive: perfume, preservative . . . | |

Preparation: Weigh in and dissolve with stirring. Bottle and add propellant gas.

The example can be repeated with the polymer components from example No. 6, 8, 9, 12, 13, 14 and 15. In each case, a foam conditioner with good properties is obtained.

### Example 8

| | |
|---|---|
| Hair gel with Aculyn 28: | [%] |

Phase 1:

| | |
|---|---|
| Polymer component from example No. 4 | 12.00 |
| (25% strength aqueous solution) | |
| Water, dist. | 37.00 |
| Aminomethylpropanol (38% strength solution) | 1.0 |
| Further additive: preservative, soluble | |
| ethoxylated silicone, perfume . . . | |

Phase 2:

| | |
|---|---|
| Aculyn 28 (1% strength aqueous suspension) | 50.00 |

### Preparation:

Phases 1 and 2 are weighed in and homogenized separately. Phase 2 is then slowly stirred into phase 1. An essentially clear, stable gel forms.

The example can be repeated with the polymer components from example No. 6, 8, 9, 12, 13, 14 and 15. In each case, a hair gel with Aculyn 28 with good properties is obtained.

### Example 9

| | |
|---|---|
| Hair gel with hydroxyethylcellulose: | [%] |

Phase 1:

| | |
|---|---|
| Polymer component from example No. 4 (25% strength solution) | 12.00 |
| Water, dist. | 30.00 |
| Further additive: preservative, soluble ethoxylated | |
| silicone, perfume . . . | |

Phase 2:

| | |
|---|---|
| Natrosol HR 250 (5% strength solution) | 50.00 |
| Hydroxyethylcellulose (Hercules) | |

### Preparation:

Phases 1 and 2 are weighed in and homogenized separately. Phase 2 is then slowly stirred into phase 1. An essentially clear, stable gel forms.

The example can be repeated with the polymer components from example No. 6, 8, 9, 12, 13, 14 and 15. In each case, a hair gel with hydroxyethylcellulose with good properties is obtained.

### Example 10

| Conditioner shampoo: | | [%] |
|---|---|---|
| A) | Texapon NSO 28% strength (sodium laureth sulfate/Henkel) | 50.00 |
| | Comperlan KS (Coamide DEA/Henkel) | 1.00 |
| | Polymer component from example No. 4 | 20.00 |
| | (25% strength aqueous solution) | |
| | q.s. Perfume oil | |
| B) | Water | 27.5 |
| | Sodium chloride | 1.5 |
| | q.s. Preservative . . . | |

### Preparation:

Phases 1 and 2 are weighed in and homogenized separately. Phase 2 is then slowly stirred into phase 1. An essentially clear, stable gel forms.

The example can be repeated with the polymer components from example No. 6, 8, 9, 12, 13, 14 and 15. In each case, a conditioner shampoo with good properties is obtained.

1. A cosmetic composition comprising
A) a polymer component which consists of one polymer or of a plurality of different polymers, comprises at least one polyurethane and where the polymer or at least one of the polymers has acid groups which are partially neutralized with at least one inorganic base and partially neutralized with at least one organic base, and
B) at least one cosmetically or pharmaceutically acceptable active ingredient or auxiliary.

2. The composition according to claim 1, where the organic base is chosen from organic amines.

3. The composition according to claim 2, where the organic base comprises at least one amine containing hydroxyl groups.

4. The composition according to claim 3, where the organic base comprises at least one amine containing hydroxyl groups which is chosen from alkanolamines, N-alkylalkanolamines, N,N-dialkylalkanolamines, dialkanolamines, N-alkyldialkanolamines, trialkanolamines and mixtures thereof.

5. The composition according to claim 3, where the organic base comprises at least one amine containing hydroxyl groups which is chosen from monoalkanolamines, N,N-dialkylethanolamines, N-alkyldiethanolamines, triethanolamine and mixtures thereof.

6. The composition according to claim 3, where the organic base comprises at least one amine containing hydroxyl groups which is chosen from 2-amino-2-methylpropanol, N-methylethanolamine, N,N-dimethylethanolamine, N-methyldiethanolamine, triethanolamine, C₈-C₁₈-alkyldiethanolamine and mixtures thereof.

7. The composition according to claim 1, where the inorganic base is chosen from alkali metal hydroxides and mixtures thereof.

8. The composition according to claim 1, where the inorganic base used is KOH.

9. The composition according to claim 1, where, for the neutralization, a polymer component with anionogenic groups is used and the anionogenic groups are neutralized to at least 40%, with at least one inorganic base.

10. The composition according to claim 1, where, for the neutralization, a polymer component with anionogenic groups is used and the anionogenic groups are neutralized to at least 1%, with at least one organic base.

11. The composition according to claim 1, where the polymer component comprises at least one polyurethane containing carboxylic acid groups or consists of at least one polyurethane containing carboxylic acid groups.

12. The composition according to claim 1 in the form of a hair-treatment composition.

13. A hair-treatment composition according to claim 12, comprising
0.2 to 10% by weight of at least one polymer component, as defined in claim 1,
40 to 99% by weight of water and/or alcohol,
0 to 45% by weight of at least one propellant gas,
0 to 20% by weight of at least one surface-active compound,
0 to 3% by weight of at least one UV absorber, and
up to 5% by weight of further constituents.

14. A method of modifying the mechanical properties of a polymer component which consists of a polymer or of a plurality of different polymers, comprises at least one polyurethane and where the polymer or at least one of the polymers has acid groups, in which, for the neutralization, at least one inorganic base and at least one organic base are used.

15. A polymer component comprising:
at least one polyurethane which has acid groups which are partially neutralized with at least one inorganic base and partially neutralized with at least one organic base, or
a mixture of at least one polyurethane which has acid groups which are neutralized at least partially with at least one inorganic base and at least one polyurethane which has acid groups which are neutralized at least partially with at least one organic base.

16. The composition according to claim 1, where the inorganic base is chosen from NaOH, KOH and mixtures thereof.

17. The composition according to claim 1, where, for the neutralization, a polymer component with anionogenic groups is used and the anionogenic groups are neutralized to at least 50%, with at least one inorganic base.

18. The composition according to claim 1, where, for the neutralization, a polymer component with anionogenic groups is used and the anionogenic groups are neutralized to at least 70%, with at least one inorganic base.

19. The composition according to claim 1, where, for the neutralization, a polymer component with anionogenic groups is used and the anionogenic groups are neutralized to at least 80%, with at least one inorganic base.

20. The composition according to claim 1, where, for the neutralization, a polymer component with anionogenic groups is used and the anionogenic groups are neutralized to at least 3%, with at least one organic base.
